(19) United States
(12) Patent Application Publication
(10) Pub. No.: US 2004/0044225 A1
(43) Pub. Date: Mar. 4, 2004

(54) Chiral and achiral synthesis of 2-acyl substituted chromanes and their derivatives

(76) Inventors: James Kanter, South San Francisco, CA (US); Charles Marlowe, Redwood City, CA (US); Anjali Pandey, Fremont, CA (US); John J.G. Mullins, San Francisco, CA (US); Robert Scarborough, Half Moon Bay, CA (US); Greg Butke, Mentor, OH (US); Barry Jacobson, Ann Arbor, MI (US); Derek Walker, Summit, NJ (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/296,875

(22) PCT Filed: Jun. 1, 2001
(86) PCT No.: PCT/US01/18016

(51) Int. Cl. C07D 335/06; C07D 307/94

(52) U.S. Cl. 549/23; 549/396

(57) ABSTRACT

Disclosed are processes for producing a (2S) or (2R) 4-oxo-chroman-2-yl acyl compounds and chroman-2-yl acyl compounds, esters or amides thereof as well as derivatives thereof. Such processes may involve chiral synthesis or achiral synthesis, preferably coupled with a resolution procedure. Such compounds, particularly (2S) or (2R) acetic acid esters, are useful intermedates for producing platelet aggregation inhibitors and/or are themselves potent platelet aggregation inhibitors. Further disclosed are processes for making derivatives of such substantially pure, or enhanced compositions of single (2R) or (2S) enantiomer intermediates or processes for producing final products or salts from such desired enantiomers.

### FIELD OF THE INVENTION

This invention relates to novel processes for producing racemic or enantiomerically enriched or substantially pure 2-acyl substituted chromone compounds, 2-acyl substituted chromane compounds and their corresponding bicyclic sulfur analogs, which are intermediates for producing platelet aggregation inhibitors and/or are themselves potent platelet aggregation inhibitors.

### BACKGROUND OF THE INVENTION

The production of 2-acyl-4-oxo-chromenes by ring closure or substituted benzene ring structures are well known in the art. One known initial process step for the production of 4-oxochromene-2-carboxylic acid, or derivatives of such acids including acid halides or esters, uses 2-hydroxy-acetophenone compounds as starting materials. (See e.g., J. Med. Chem., Vol. 15, No. 8, 1972.) The reaction scheme to produce the 2-carboxylic acid and esters is as follows:

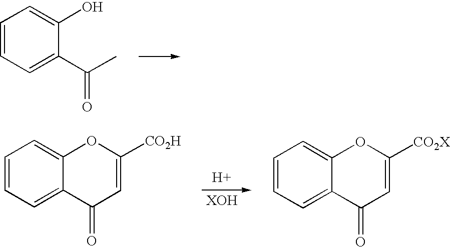

where X is ethyl, for example. The acid can be converted to an acyl halide, such as acyl chloride, instead of the ethyl ester by reacting it with SOCl₂, for example. A further reaction of the acyl chloride with NH₃ can be used to produce the carboxamide.

The above chromene-derivative compounds have been reported as useful intermediates for the production of compounds wherein the phenyl ring of the chromene ring structure is further substituted by a benzoylamino derivative to produce antidepressants. See, for example, U.S. Pat. No. 5,659,051.

Further, when the production of a 2-chroman-2-yl acetic acid compound racemic starting material is desired, such can be accomplished by hydrogenation of a coumarin derivative with a reducing agent under standard reduction conditions or via hydrogenation conditions by using standard catalysts selected from the group comprising diisoamylborane, lithium tri-butoxyaluminohydride, lithium triethylborohydride, lithium trimethoxyaluminium hydride, sodium borohydride, H₂/Pd/C, and the like. Such catalysts and procedures may be utilized to hydrogenate the double bond in the lactone ring and/or replace the keto group with a hydroxyl group. In a preferred aspect lithium tri-butoxyaluminohydride, LiAlH₄, or diisoamylborane (DIABO) may be used as part of such a process step to reduce the two position keto group to a hydroxyl group.

The hydroxyl group may then be replaced by an acetic acid side chain by a standard chain extension/replacement reaction of the alcohol intermediate. For example, reaction of the alcohol intermediate with chloroacetate under basic conditions in the presence of pyridine will result in an acetic acid side chain in the two position.

### SUMMARY OF THE INVENTION

The present invention relates to novel processes for producing achiraJ bicyclic intermediates or intermediates which are a substantially pure single enantiomer or a composition substantially enriched in a single enantiomer of chromans substituted at the 2-position with C₁-C₈-acyl (branched and straight-chained) groups, such as 2-carboxylic acid compounds, chroman-2-yl acetic acid and propanoic acid, as well as other derivatives and sulfur analogs thereof, such as esters, which are intermediates for producing therapeutic agents, or are themselves therapeutic agents, for disease states in mammals that have disorders caused by or impacted by platelet dependent narrowing of the blood supply.

In accordance with one preferred embodiment, there is provided a process for making a bicyclic compound, or a pharmaceutically acceptable salt of such compound, according to the following formula:

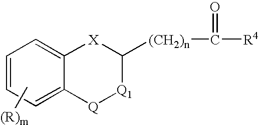

wherein:

n is 0 to 6;

X is O or S;

m is 1 to 4;

R are independently selected from the group consisting of alkyl, alkoxy, lower alkenyl, hydroxy, thio, amino, substituted amino, nitro, halo, and CF₃;

R⁴ is selected from the group consisting of hydroxy, alkoxy, alkenyloxy, halogen, amino, and substituted amino, and

each of Q and Q₁ are independently selected from the —C(—R², —R³)—, wherein each of R² and R³ is independently selected from the group consisting of alkyl, alkoxy, alkenyl, hydroxy, thio, nitro, halo, and CF₃, or R² and/or R³ together with the carbon to which it is attached form a carbonyl group. The method comprises (a) and (b):

(a) combining a first compound having the formula:

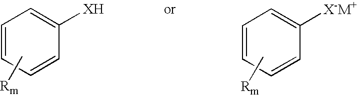

wherein M⁺ is a metal ion; with a second compound having the formula R⁴—Q—Q₁—CH(Z)—(CH₂)ₙ—C(═O)—R¹ wherein Z is halo or OH, optionally in the presence of a Walden catalyst; and

(b) cyclizing the product of (a) to form a bicyclic compound.

In preferred embodiments, the Walden catalyst in (a) is selected from the group consisting of PCl₅, PBr₃, PI₃, PF₃, SOCl₂, KOH and Ag₂O; and (b) comprises use of a basic Walden catalyst or a Friedel Crafts catalyst, wherein the catalyst in (b) is preferably one selected from the group consisting of AlCl₃, NaOH, KOH, sodium carbonate, and potassium carbonate.

In preferred embodiments, the second compound is a single (R) or (S) enantiomer, and the compounds formed are either a (2S) or (2R) compound, or an enriched (2S>2R) or (2R>S) mixture wherein one of the (2S) or (2R) is present at greater than about 70%.

In preferred embodiments, the second compound is a mono or diester of 3-halomalonic acid, or a mono or diester of 3-hydroxymalonic acid; Z is oxygen; m is 0 or 1; and R is amino or nitro.

In accordance with other preferred embodiments, step (a) of the foregoing process produces a compound having the formula:

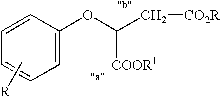

wherein R¹ is hydrogen or alkyl, and step (b) comprises:

(b1) acylating the compound from step (a) at chain “a” to form an acyl halide;

(b2) displacing the halide with ammonia to form an amido group; and

(b3) performing a Friedel-Crafts cyclization to form a compound having the formula:

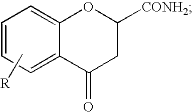

and may further comprise hydrolyzing the amido group at the 2-position to form an acid; and performing a chain extension to form a compound having the formula:

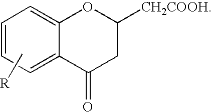

Alternatively, step (b) comprises:

(b1) reducing at chain “a” to form a methanol group;

(b2) displacing the methanol hydroxyl group with a cyano group and acidifying to form an acetic acid group;

(b3) performing a Friedel-Crafts cyclization to form a compound having the formula:

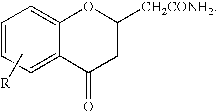

and may further comprise hydrolyzing the amide to form a compound having the formula:

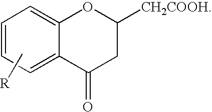

Alternatively, step (b) comprises:

(b1) acylating the compound from step (a) at both chains “a” and “b” to form acyl halides;

(b2) performing a Friedel-Crafts cyclization to form a compound having the formula:

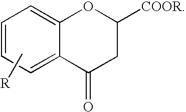

Alternatively, step (b) comprises heating the compound from step (a) in the presence of a strong base to form a compound having the formula:

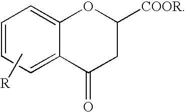

wherein either of the last two alternatives may further comprise performing a chain extension to form a compound having the formula:

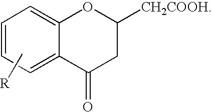

In preferred embodiments, the foregoing compounds may be nitrated if amino or nitro is not already present and/or the compounds reduced to remove the 4-oxo group and reduce the nitro group to amino (if present). The reduction may take place in one or more steps. If an acid is formed, further reaction may be done to obtain a corresponding ester, preferably the ethyl ester.

In a further preferred embodiment, a mono or diester of 3-halomalonic acid, or a mono or diester of 3-hydroxymalonic acid is reacted with 4-hydroxy-3-methylaniline and then cyclized using a strong base. The method may further comprise reducing the compound formed to remove the 3-oxo group.

In a further preferred embodiment, there is provided a process of making a compound according to the formula:

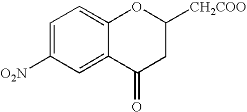

The process comprises reacting a 4-nitrophenoxide compound with a malonic ester compound having the formula:

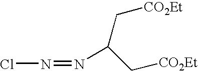

or its corresponding anhydride, and performing a Friedel-Crafts cyclization reaction. If the corresponding anhydride is used, there is an additional step of heating the adduct of the anhydride and 4-nitrophenoxide in water to provide the diacid prior to the cyclization. Either process may further comprise reducing (removing) the 4-oxo group and reducing the nitro group to amino. The reduction may be accomplished in one step, or in multiple steps.

In accordance with another preferred embodiment, there is provided a process for making a compound according to the formula:

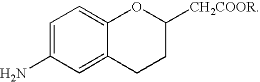

The process comprises

(a) reacting 4-nitrophenol and maleic acid in methane sulfonic acid;

(b1) performing a chain extension to lengthen the chain at the 2-position; and

(b2) reducing the 4-oxo and 6-nitro groups;

wherein R¹ is hydrogen or alkyl, and (b1) and (b2) may be performed in either order.

In accordance with preferred embodiments, compounds having one of the following three formulae are produced according to the methods herein:

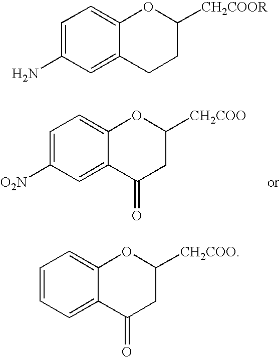

### DETAILED DESCRIPTION OF THE PREFERRED EMBODIMENTS

The terms “alkyl” and “lower alkyl” as used herein refers to a monovalent straight or branched chain radical of from one to about 10 carbon atoms, with one to about six being preferred, including, but not limited to methyl, ethyl, n-propyl, isopropyl, n-butyl, isobutyl, tert-butyl, n-hexyl, and the like.

The term, “substituted alkyl” as used herein refers to an alkyl group as just defined, substituted by one or more groups selected from halo, amino, hydroxyl, substiuted amino, nitro, and the like. Examples of such groups include chloromethyl, hydroxyethyl, aminomethyl, and the like.

The terms “halo” or “halogen” as used herein refer to F, Cl, Br, or I substituents.

The term, “acyl” as used herein refers to a group having the formula R—X—C(═O)— wherein R is preferably alkyl, substituted alkyl, or alkenyl, and X is 0 or a bond.

The terms “alkenyl” and “lower alkenyl” as used herein refers to a monovalent straight or branched chain radical of from two to six carbon atoms containing a carbon double bond including, but not limited to, 1-propenyl, 2-propenyl, 2-methyl-1-propenyl, 1-butenyl, 2-butenyl and the like.

The terms “alkoxy” and “alkenyloxy” as used herein refer to alkyl and alkenyl groups as defined above, respectively, which are linked through an oxygen atom, including, but not limited to, —OCH3, —OCH2CH3, —OCH═CH2 and the like.

The term “alkylthio” as used herein is an alkoxy group in which the 0 has been replaced by S.

The term “substituted amino” refers to an amino group in which one or more hydrogens have been replaced by alkyl, substituted alkyl, alkenyl or similar groups.

There is a need for improved processes for producing resolved substantially pure enantiomer compositions or compositions that are enhanced in concentration with respect to a single enantiomer, as well as improved intermediate compounds that are useful as intermediates in processes for producing platelet aggregation inhibitors having the phenyl ring of the chromanes substituted differently than for the antidepressant compounds reported by U.S. Pat. No. 5,659,051, and further having the 4-oxo group replaced by hydrogen atoms as well as having the double bond removed from the 2-3 positions of the pyran ring by saturation (i.e., hydrogenation).

There is also a need for producing achiral and chiral intermediates which can be used to produce the bicyclic compound described in U.S. Pat. No. 5,731,324. The chromane carboxylic acid derivatives can be resolved into racemic mixtures (R/S) that are enriched with one of the R or S enantiomers, or resolved to produce substantially pure (e.g. preferably at least 90% enantiomerically pure, more preferably at least 93%, at least 95%, at least 97%, and at least 99% enantiomerically pure) compositions of a single enantiomer (R or S enantiomer). Due to inherent losses of up to 50% or more of the starting materials (assuming a 50/50 R/S racemate) during enantiomer resolution, there is a need for a process which efficient produces large quantities of enriched enantiomer compositions or a single enantiomer, preferably from a simple chiral starting material. Such would reduce or avoid the inherent losses of material and expenses related resolving an racemate.

Oftentimes, two compounds having the same molecular formula will have a different spatial relationship of the atoms around a “chiral” carbon atom. In some circumstances, it is desired to have a pure or enriched composition of one of the two (R or S) enantiomers. One or more of the biological activity, physical properties or chemical properties often differ between the enantiomers to make it such that one enantiomer is a preferred structure. In fact, in nature sometimes only one of the two chiral structures is made. For example, (−)-2-methyl-1-butanol is formed in yeast fermentation of starches and only (−)-malic acid (an asymmetrical alpha-hydroxy diacid compound, HOOC—CH(OH)— CH—COOH) is present in fruit juices. While some simple chiral compounds are available in nature, others may be produced synthetically or may be obtained commercially.

There is a need to provide an improved process which produces a single enantiomer or a composition that is substantially enriched with one enantiomer with respect to 4-oxo-2-acyl-chromane and 2-acyl-chromane compounds, as well as derivatives thereof, and intermediates useful for producing such compounds. In particular, an improved method is needed for making such (R or S) enantiomer intermediate 2-carboxylic acid ester chromane compounds that are useful for producing compounds in the antithrombotic field.

One or more of the foregoing needs may be met using the processes described herein and the compounds and intermediates made thereby.

In accordance with one class of preferred embodiments, there is provided a process that utilizes an asymmetrical alpha-hydroxy diacid enantiomer and a phenol derivative as starting materials. Since the acid group that is closer to the carbon atom which is substituted by the hydroxyl group tends to be far more acidic (alpha hydroxy acid group) and more reactive than the more distant acid group (methylene acid group), selective reactions may be used to substantially produce the single desired enantiomer with respect to the chiral 2-position carbon in the ring that is substituted by the acyl group. Preferably the alpha hydroxy diacid enantiomer is the (R or S) enantiomer of alpha-hydroxy butanoic acid (malic acid). As pointed out above, a single enantiomer (S), also referred to as (−), of malic acid occurs naturally in fruit juices, but each of the two enantiomers are available separately from commercial suppliers, such as from Aldrich Fine Chemicals. Regardless of whether the enantiomer is the (R) or (S) enantiomer, reaction steps can be selected with respect to the alpha-hydroxy acid group or to the methylene acid group to produce either of the desired (R) or (S) enantiomer 2-acyl chromane compounds. Preferably, the natural malic acid such as from fruit juices is used as the reactant since it tends to be less expensive.

In one embodiment, there is a compound and a process for making the compound (or a salt thereof) according to the formula,

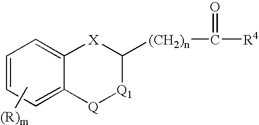

wherein:

n is 0 to 6;

X is O or S;

R is independently selected from the group consisting of alkyl, alkoxy, alkenyl, hydroxy, thio, amino, substituted amino, nitro, halo, and CF₃;

m is 0 to 4; (although R₀₋₄ may be used as shorthand for Rₘ where m is 0-4)

R⁴ is a member selected from the group consisting of hydroxy, alkoxy, thioalkyl, —SH, alkenyloxy, halogen, amino and substituted amino, and

each of Q and Q₁ are independently selected from the —C(—R², —R³)—, wherein each of R² and R³ is independently selected from the group consisting of hydrogen, alkyl, alkoxy, alkenyl, amino, hydroxy, thio, nitro, halo, and CF₃, or R² and/or R³ together with the carbon to which they are attached form a carbonyl group.

Examples of procedures to make such compounds are in Schemes I through XIX and the Examples which follow. Some of the Schemes involve chiral synthetic techniques and some form racemates which may be resolved, if desired. In a preferred procedure, a phenol or thiophenol derivative is reacted with a chiral or achiral compound R⁴—Q—Q₁—CH(halogen)—(CH₂)ₙ—C(═O)—R⁴ (wherein R⁴ is a member selected from the group consisting of H, —OH, —SH, halo, amino, substituted amino, thioalkyl, alkoxy, or a similar ring condensation leaving group), in the presence of a Walden catalyst, preferably, NaOH, KOH, sodium carbonate, potassium carbonate, and the like, to provide an ether or thio ether, which is then cyclized to form the bicyclic compound. In a further preferred embodiment, the phenol or thiophenol is a phenoxide or thiophenoxide derivative and is reacted with a chiral halogen compound ((S) or (R) enantiomer with respect to the carbon attached to the halogen atom) to produce a substantially (S) or (R) ether. Even further preferred is such a reaction which produces exclusively an (S) or (R) ether.

In one preferred embodiment, there is provided a process for making such a compound, comprising reacting a derivative of phenol, thiophenol, sodium phenoxide, sodium thiophenoxide, and the like, with an the alcohol of the derivative of a chiral hydroxy diacid (or with a derivative of the hydroxy group such as the halide which has displaced the hydroxy groupby virtue of a Walden catalyst without producing a racemate) in the presence of a Walden Inversion Catalyst (preferably NaOH, KOH, sodium carbonate, potassium carbonate, and the like) or after the alpha-hydroxy diacid has been treated with the Walden Inversion Catalyst to form a halide (see catalyst examples below) as follows:

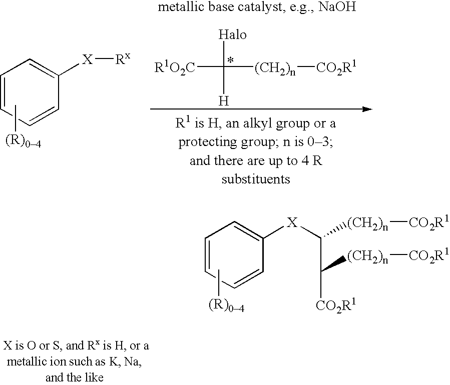

wherein R is substituent on the phenyl or benzene ring (replacing a hydrogen atom on the ring) such as an amino group (or a protected amino group such as a benzamido or acetamido group) or a group that can be converted to an amino group (e.g., hydrogen, halogen, nitro, and the like), and R¹ is hydrogen, the alkyl core of an alcohol group which can form an ester such as a methylene or ethylene group from methanol or ethanol, or (whether alone or together with its neighboring oxygen atom is a protecting group). Also noted above, the catalyst for the reaction is a Walden Inversion Catalyst, preferably selected from the group consisting of PCl₅, PBr₃, PI₃, PF₃, SOCl₂, KOH or Ag₂O, or the like, or a combination thereof. Most preferably, the Walden catalysts are PBr₃ and KOH, wherein the alcohol has been treated with PBr₃ under conditions to change the (R or S) enantiomer alcohol into its opposing (S or R) alpha bromide diacid or diester, and is then reacted with the phenol in the presence of the Walden Inversion Catalyst KOH to form a phenoxy derivative. Some of the Walden catalysts may preserve the (R ) or (S) geometry of the alcohol starting material with respect to the carbon to which the alcohol group is attached, while some members of the catalyst group (such as the phosphorus halides) may cause inversion of the geometry (e.g., (S) may be converted to (R), or the inverse). However, since the synthesis can proceed to ring closure from either of the acid groups, the desired geometry for the ring 2 position of the bicyclic ring can still be obtained.

It should be noted that in the structure shown above, both solid and hashed-bonded groups are shown. In such circumstances, this should be understood to represent that, depending upon the reactants chosen, either the (R) or (S) form may be pure form. Alternatively, where there is a chiral center having a particular sterochemistry (not a racemate) it may also be indicated be a (*) next to the chiral carbon, or only one of the enantiomers may be shown in the structure.

Preferably, the methylene acid group is esterified and the alpha hydroxy acid group is a —COOH group or can be converted into such a compound. If the more reactive alpha-hydroxy acid group needs protecting while the methylene acid group is further reacted, it may be converted to a carboxamide group, for example, by selectively reacting it with SOCl₂, followed by reaction with NH₃ under conditions such that the methylene acid (or esterified methylene acid) does not react. Since the alpha-hydroxy acid group is about 100 times more reactive than the methylene acid group, such a reaction is readily accomplished.

For example, the carboxamide is formed as follows:

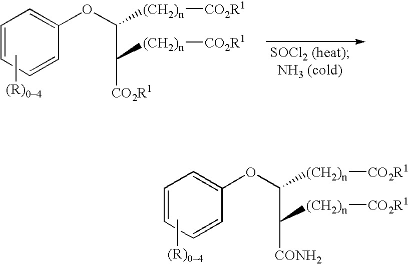

wherein the acyl halide (acid chloride) is first formed and readily converted to the carboxamide.

The methylene acid group can then be optionally acidified, if desired, to remove the ester group. The methylene acid group can be cyclized with the phenyl ring without disturbing the chiral carbon adjacent to the oxygen atom. For example, when the methylene acid group side chain is an acetic acid side chain (malic acid starting material) then a Friedel Crafts reaction can be used to form a 4-oxo-2-carboxamide chromane derivative.

A preferred Friedel Crafts reaction may be illustrated as follows:

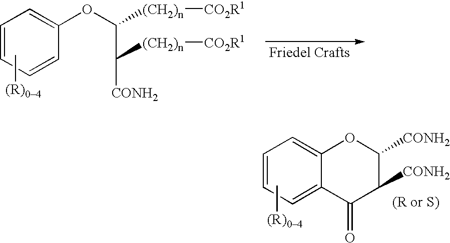

wherein each of the (R or S) 2-carboxamide enantiomers is possible, depending upon whether the (R or S) enantiomer of malic acid is used as the starting material. Moreover, by choosing the desired Walden Inversion catalyst, a particular (R ) or (S) enantiomer of the alcohol starting material can be converted to the desired enantiomer prior to reacting it with the phenol.

In any event the 2-carboxamide enantiomer obtained may be reacted further in several ways. Such preferred ways including convertintg the 2-carboxamide group to a carboxylic acid group by reacting the compound with sulfuric acid and water, or an ester can be formed by then adding an alcohol such as methanol or ethanol. Likewise, the carboxamide, acid, or ester chromanone can be reduced to a chromane by hydrogenation, such as by using H₂/Pd/C and other known hydrogenation agents. Standard reaction conditions can be utilized for such steps.

A preferred embodiment involving formation of the acid, then formation of the ethyl ester, followed by reducing the chromanone to a chromane derivative is illustrated as follows:

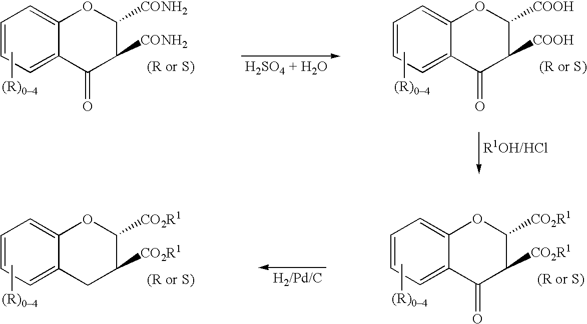

wherein it is also possible to change or add the R groups prior to one of the above steps. If a 6-position amino group is desired and the phenyl ring is unsubstituted, reaction of the chromanone compound with HNO₃ will result in the —NO₂ group being directed in high yield to the 6-position. During the subsequent hydrogenation step or steps, the —NO₂ group may be converted to an amino group and the 4-oxo group may be removed.

If desired, the 2-position carboxamide side chain can be extended to form a longer chain, such as an acetic acid (or acetic acid ester) side chain or a derivative thereof without disturbing the chiral carbon at the two position. A preferred process for making the acetic acid chain is as follows. The carboxamide group is first converted to the acid group and then to the carboxylic acid ester as shown above. The ester group is reduced to the alcohol by using standard reduction or hydrogenation conditions and/or catalysts, for example diisoamylborane, lithium tri-butoxyaluminohydride, lithium triethylborohydride, lithium trimethoxyaluminium hydride, sodium borohydride, and the like. In a preferred aspect, lithium tri-butoxyaluminohydride, LiAlH₄, or dilsoamylborane (DIABO) may be used as part of such a process step to reduce the two position ester group to a methanol group. After formation of the 2-methanol group, it can be reacted with an cyanide compound such as CuCN or KCN to extend the carbon chain, followed by an acid such as HCl in water or alcohol to convert the cyano group to an acid group. Optionally, the acid group may be reacted with an alcohol under acidic conditions to form an ester. These reactions, starting from the ester compound shown above, may be illustrated as follows:

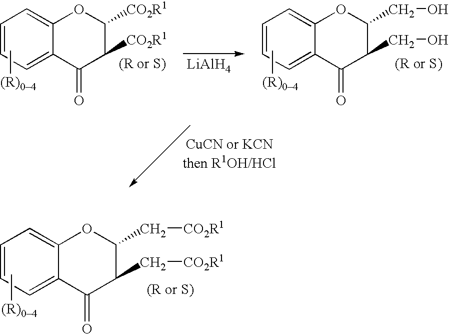

Further, as described above for the 2-acyl side chain chromanone compound, the compound formed above may also be reduced in the same manner to form the chromane enantiomer. Likewise, it is also possible to change or add the R groups prior to one of the above steps. If a 6-position amino group is desired and the phenyl ring is unsubstituted, the chromanone compound may be reacted with HNO₃ such that the —NO₂ group is directed in high yield to the 6-position. During the hydrogenation step shown below, the —NO₂ group, if present, is preferably converted to an amino group while the 4-oxo group is removed. This reaction may be shown as follows:

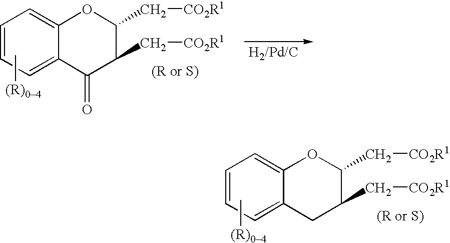

wherein the resulting chromane is a (2S) or (2R) enantiomer depending upon whether the (R) or (S) enantiomer of the alpha hydroxy alcohol starting material is used.

While the above reaction description relates to the ring closure using the methylene acid group of the alcohol diacid after it has reacted with a phenol, it is possible to protect the methylene acid group and proceed to close the ring using the alpha-hydroxy acid group portion. In a preferred embodiment of such a ring closure case, the opposite enantiomer is obtained from the same alpha-hydroxy diacid than is obtained by ring closure which uses the methylene acid group. After reaction of the alpha-hydroxy diacid with the phenol compound to form an initial intermediate as shown above, both acid groups are esterified. The more reactive alpha-hydroxy acid ester group is selectively reduced to the aldehyde or alcohol, preferably to the aldehyde. The methylene acid ester group is then converted to the acid chloride and subsequently to its carboxamide, using the acid chloride and carboxamide formation steps that were described and shown above for the alpha-hydroxy acid group.

The reduction steps and carboxamide formation steps of the aforementioned preferred embodiment may be illustrated as follows:

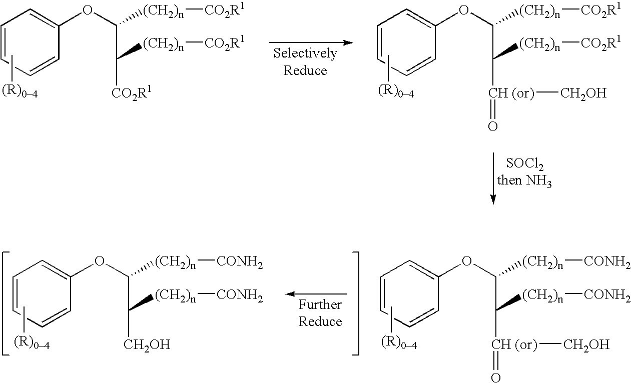

where the above step which is enclosed in square brackets is an optional step which is not required if the first selective reduction formed the alcohol instead of the aldehyde. Once the methylene acid group has been protected by converting it to a carboxamide group, the alcohol can be further reacted to extend its chain by an additional carbon and converted to an acid group. Such reactions are essentially the same as the above CuCN or KCN step followed by acidification used and shown above for the carboxylic acid side chain extension that was done after ring closure. However, in this case the alcohol group is extended prior to ring closure, which makes it possible to use a Friedel Crafts ring closure reaction to form a 6-membered ring. Once the ring is closed, the geometry at the 2-position is fixed and the terminal carboxamide group (amino group that protected this chain from being involved in the ring closure step) can be readily converted to the acid group and then to an ester if desired.

Those steps may be illustrated as follows:

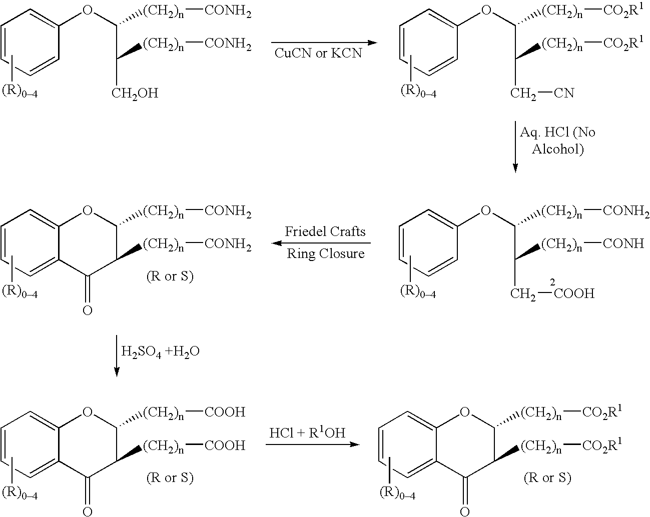

as in the above discussion, the phenyl ring substituents, R, may be adjusted or changed and the enantiomeric chromanone compound can be converted to its corresponding chromane compound. The reaction steps may be illustrated as follows:

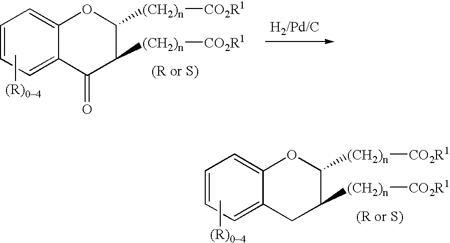

if the acid side chain converted to the free acid, it may be re-esterified with an alcohol and a mineral acid such as EtOH and HCl to yield the desired ester.

When the phenyl ring of the bicyclic core does not contain a nitro or amino group, the bicyclic ring structure may be optionally formed without adding the nitro group to the starting material. The production of a 6-position nitrogen substituent from the bicyclic (R) or (S) chromanone enantiomer intermediate may be depicted as follows:

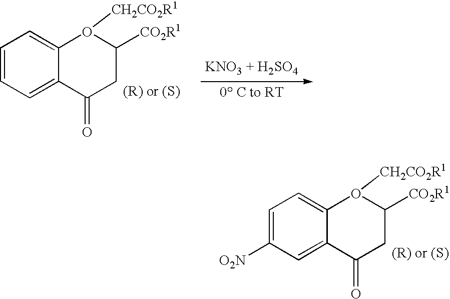

wherein, as exemplified the predominant placement of the nitro group is at the 6-position as shown. Other nitration procedures may be utilized such as bromination followed by substitution of the nitro group for the bromine group, for example. However, the above nitration procedure is straight-forward and is preferred.

Although the step above, and some of which follow, depict racemates, it is to be understood that such reactions may also proceed with enantiomerically enriched or subtantially enantiomerically pure compounds.

As pointed out above and in one or more schemes above, the chromane nucleus core can be produced from the above ester by reducing the 4-position oxo group, which reduction may also hydrogenate an —NO₂ group on the phenyl portion of the chromanone to its —NH₂ form, unless it is protected with a suitable protecting group, such as benzoylamino group, or the like or unless a selective reagent or conditions are used to promote reduction of one group over the other. Standard reduction or hydrogenation conditions and catalysts may be utilized, for example diisoamylborane, lithium tri-butoxyaluminohydride, lithium triethylborohydride, lithium trimethoxyaluminium hydride, sodium borohydride, H₂/Pd/C, and the like, may be utilized to hydrogenate the double bond and/or replace the keto group with a hydrogen atom. In a preferred aspect lithium tri-butoxyaluminohydride, LiAlH₄, or diisoamylborane (DIABO) may be used.

The following reaction steps show the conversion of 2-carboxylic acid compound to an acyl halide (chloride in this case) and replacement of the halogen atom with a cyano group, which can then be converted in a later step to an carboxylic acid group to result in an acetic acid side chain:

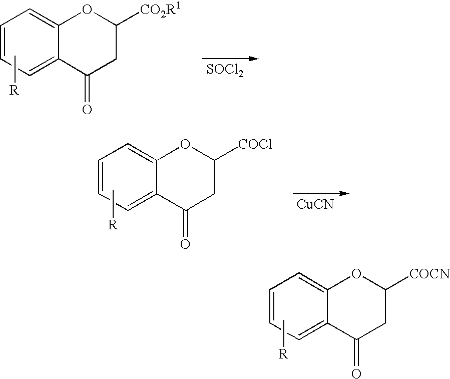

wherein the preferred R group is an NO₂ group which can be later converted to an amino group, such group preferably being in the 6 position on the bicyclic ring structure. The 2-acyl-cyanate group can be hydrolyzed to an alpha keto carboxylic acid group by exposure to a strong mineral acid (such as HCl) at ambient temperature as follows:

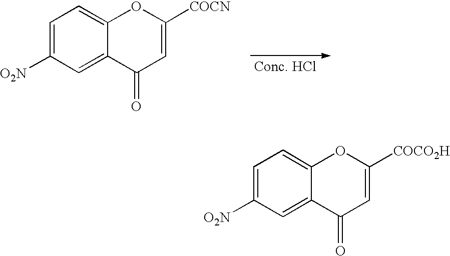

to provide a good yield of the alpha keto acetic acid group which is now ready for a hydrogenation step. Other known methods of extending the chain from a carboxylic acid to an acetic acid may also be used, including those shown herein.

Preferably the two keto groups (one ring, one pendant), along with the nitro group and the unsaturated bond on the pyran ring, if present, are all removed by hydrogenation in a single step which may be monitored by HPLC until the reaction is complete. However, in one embodiment, the hydrogenation is conducted in two steps. The first step is hydrogenation under mild conditions in the presence of an alcoholic solution (such as methanol, ethanol or the like), or in ethyl acetate solution or the like, at about 30 psi of hydrogen for about an hour or two followed by purging the bomb with nitrogen then hydrogen. In the second step, glacial acetic acid is added to the alcoholic solution of the bomb, the hydrogen pressure is increased to 45-65 psi and the temperature raised to between 50° C. to 95° C. maintained until the hydrogenation is complete by monitoring of HPLC. While hydrogenation may be visualized as either a one-step or two-step process, it is still a one-pot non-separation step. The overall reaction for a preferred single or multi-step hydrogenation process may be exemplified as follows:

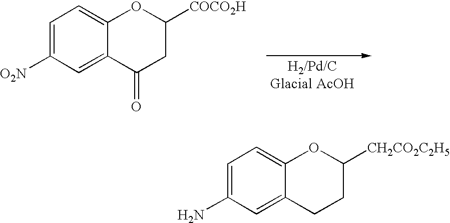

This process will provide a good yield of the racemate or a single enantiomer based upon the starting compound and the synthetic route chosen.

If desired, the amino group such as is formed above, can be converted to a salt by using a strong mineral acid such as H₂SO₄ in an alcohol such as ethanol, followed by HCl in an alcohol such as ethanol to produce the desired ester group and the mineral acid salt of the amine, for example, as follows:

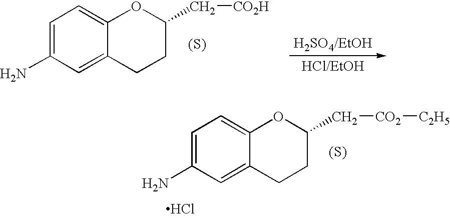

wherein the preferred ethyl ester of the (S)-enantiomer is shown as being produced after the hydrolysis step and HCl is utilized as the acidic solution for the esterlfication to cause the amino group to form a hydrochloride salt, but other esters such as the methyl or propyl ester may likewise be envisioned. Such esters may be formed directly such as by selecting the appropriate alcohol, or they may be obtained by transesterification. Salts other than hydrocloride, including, but not limited to, tartrate, glutamate, lactate, hydrobromide, and other such parmaceutically acceptable salts.

Hydrogenation may also reduce the 2-position side chain to hydroxymethyl. In such a case, the side chain may be extended into an acetic acid group, or into an acetic acid ester group, as follows:

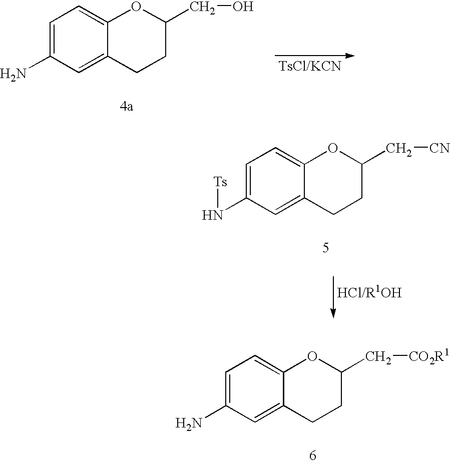

wherein R1 is preferably an ethyl ester group. Such reaction may proceed on a racemate or on an enriched or substantially purified single enantiomer, whether such material is obtained from a resolution process or formed directly using chiral synthetic techniques. As shown in the above reaction steps, when the amino group is unprotected, a tosyl group may be added to the amino group during the same step in which the cyano group replaces the alcohol group. In such a case, during the conversion of the cyano group to a carboxylic acid group an excess of concentrated HCl can be used to ensure that the tosyl group is removed from the amino group to yield a —NH₂ group. Alternatively, after removal of the HCl from the reaction mixture and obtaining the 2-chromane compound, such as by neutralization of the solution and recrystallization of the compound in isopropyl alcohol, for example, a solution of a more nucleophilic acid such as HBr may be used to remove the tosyl group from the amino group. However, under such conditions care should be taken to avoid replacement of the —OH of the carboxylic acid group with a bromine atom to yield an acylbromide.

If desired, the ester group can be modified with a group which can be utilized to resolve the R or S enantiomers, such as a conventional camphor sulphonic acid derivative, a dibenzoyl tartaric acid derivative, and the like, or by hydrolyzing the ester to an acid with a selective lipase to produce a desired enantiomer, for example, as follows:

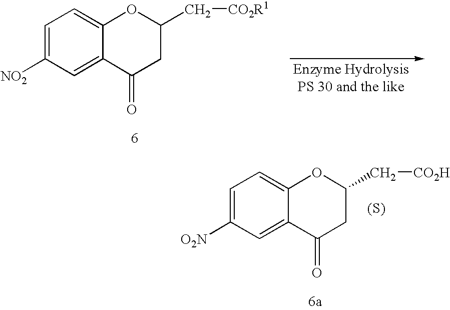

wherein the preferred ethyl ester of the S-enantiomer is shown as being produced after the hydrolysis step and HCl is utilized as the acidic solution for the esterification to cause the amino group to form a hydrochloride salt. Other esters such as the methyl or propyl ester may likewise be envisioned.

In a preferred aspect the efficiency of the enzymatic resolution may be increased by having a 6-nitro group prior to enzymatic resolution and then converting the 6-nitro group to an amino acid group after the enzymatic resolution. In a preferred aspect, the efficiency of the enantiomer resolution step is increased by recycling the R-enantiomer by racemization via subjecting it to at least one ring-opening and closure as a racemization step followed by further resolution of the thus generated racemate to increase the amount of S-enantiomer obtained. The preferred solvent for recrystallization is methanol or isopropyl alcohol. In either event, after resolving the S-enantiomer and obtaining the 2-carboxylic acid form of the molecule, HCl and ethanol may be added to the compound to form the 2-carboxylic acid ethyl ester and an excess of HCl is then utilized to produce the hydrochloride salt of the amino group. A preferred process of resolution using liase is discussed in greater detail later herein.

In accordance with a preferred embodiment, there is provided a process that uses 4-nitrophenol to produce ethyl 2-(6-amino-chroman-2-yl)acetate 7 as set forth in Scheme XVII which follows on a later page.

Referring to Scheme XVII, the individual preferred reaction steps illustrated therein will now be discussed in more detail. In the first step, there is a cyclization reaction wherein maleic acid and a phenol react under acidic conditions producing a 4-chromanone. A preferred phenol is 4-nitrophenol. An example of this reaction is as follows:

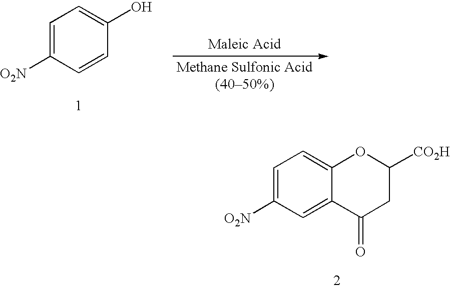

In a preferred embodiment, the maleic acid is present in about a 2:1 mole ratio with respect to the phenol. The cyclization is performed at about 75-95° C. for about 15-30 h, preferably, at 92° C. for about 20 h. The reaction mixture is then cooled; the solid is isolated; the solid is washed with water, a dilute base, and then again with water to provide compound 2 as the product as a yellow solid at about 40-50% yield.

In a further step, the carboxylic acid of compound 2 is converted to an intermediate for converting the carboxylic acid more easily into other derivatives. Preferably, compound 2 is converted to an acyl chloride 3 by reaction with thionyl chloride, as follows:

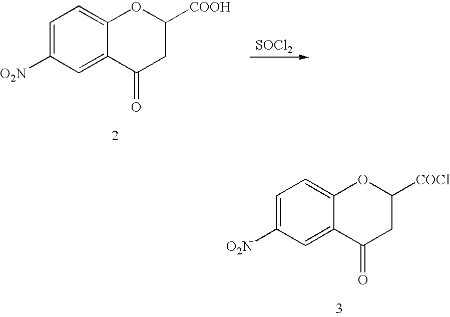

In another two steps, compound 3 is converted into compound 4 with nucleophilic attack of cyanide ion on the chloride of the acyl chloride 3. The resulting compound 4 is hydrolyzed in acidic conditions to yield α-keto carboxylic acid 5, as shown below.

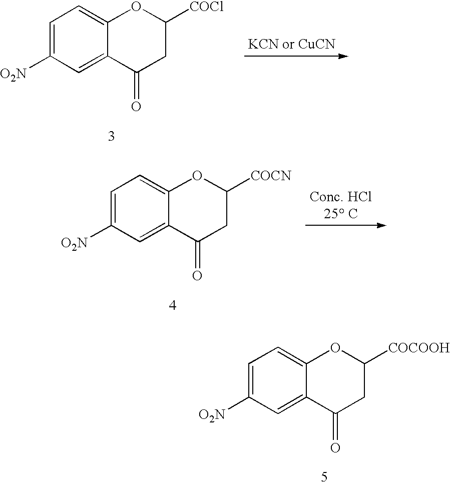

Then, compound 5 undergoes hydrogenation to convert the nitro group into an amino group. Examples of conditions to reduce the nitro group include catalytic hydrogenation and use of chemical reducing agents in acidic solutions. A preferred condition for this conversion is the use of hydrogen in the presence of catalytic palladium/carbon and glacial acetic acid. Then, compound 6 is esterified in acidic ethanol to yield compound 7, as shown below.

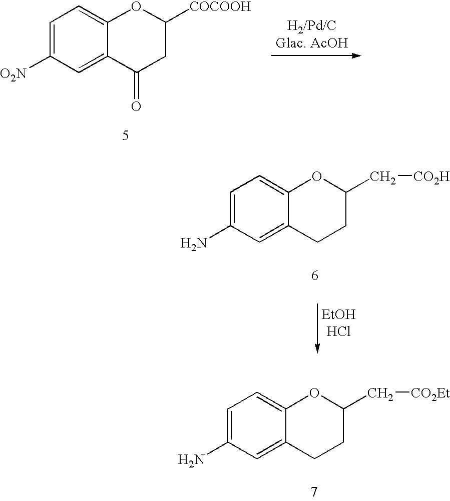

While the ethyl group was used to form the ester of the acetic acid side chain in the last step, the ethyl group can be replaced by hydrogen or another group capable of forming an ester selected from lower alkyl, lower alkenyl, lower alkynyl, phenyl, cinnamyl or other ester groups. Optionally, the amino group can be protonated to isolate the product as an amine acid halide salt or the like.

In accordance with another preferred embodiment, there is provided a process that uses 4-nitrophenol to produce ethyl 2-(6-amino-chroman-2-yl)acetate 7 as set forth in Scheme XVIII which follows on a later page.

Referring to Scheme XVIII, the individual preferred reaction steps illustrated therein will now be discussed in more detail. In the first step, there is a cyclization reaction wherein maleic acid and a phenol react under acidic conditions producing a 4-chromanone, as shown above in reference to Scheme XVII. A preferred phenol is 4-nitrophenol to provide compound 3 as the product. To the yellow solid is added a mixture of H₂SO₄/EtOH, preferably at room temperature, to form an ester 8. An organic solution containing the ester 8 is washed with water and brine, dried over magnesium sulfate, and concentrated under vacuum to yield the purified product. Typically, the overall yield of this process is about 85-95%.

In further steps, the reaction preferably proceeds by reducing conditions which affect the keto, ester or acid, and nitro groups of compound 8 to yield compound 10, as shown below:

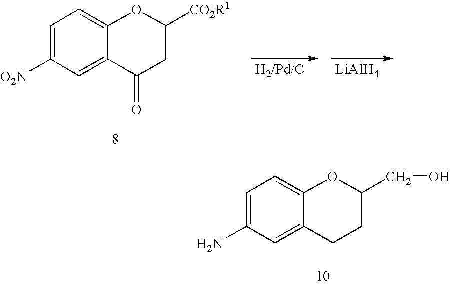

The nitro group of compound 8 is reduced to an amino group. Reduction may be accomplished by catalytic hydrogenation or by the use of chemical reducing agents in acidic solution. An example of a reducing agent for this conversion is hydrogen in the presence of catalytic palladium/carbon.

Then, reducing the 4-keto group of the chromanone ring system produces a chromane ring system. Examples of reducing agents for converting a ketone group to a methylene group are lithium aluminum hydride, lithium tri-butoxyaluminohydride, sodium borohydride, H₂/Pd/C, and the like. Preferably, these conditions may also be used to reduce the ester group to a hydroxymethyl group. In a preferred aspect, lithium aluminum hydride, lithium tri-butoxyaluminohydride, or sodium borohydride would reduce both the ketone and the carboxylate ester at the 2-position to a hydroxymethyl group to yield compound 10.

Compound 10 is further modified by converting the 2-hydroxymethyl into a chromanylacetic ester 7 thereof, as follows:

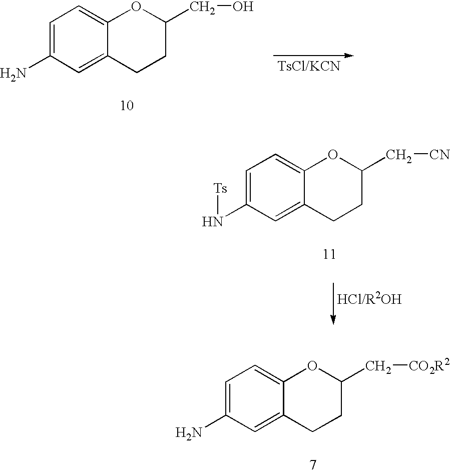

As shown above, a tosyl group is added to the amine and a cyanide group replaces the hydroxyl group in one pot to yield compound 11. In a subsequent step, the tosyl group may be removed concomitant with the hydrolysis of the nitrile to a carboxylate with excess concentrated hydrochloric acid. Alternatively, after nitrile hydrolysis with hydrochloric acid, the hydrochloric acid may be removed from the reaction mixture and recrystallization of the product from isopropyl alcohol. Then, a more nucleophilic acid such as HBr may be used to remove the tosyl group. However, care must be taken to avoid converting the carboxylic acid into an acyl bromide. Then, compound 11 is esterified in the presence of an alcohol and an acid to yield compound 7. Preferably, R² is an ethyl group that results from acidic ethanol.

While the ethyl group was used to form the ester of the acetic acid side chain in the last step, the ethyl group can be replaced by hydrogen or another group capable of forming an ester selected from lower alkyl, lower alkenyl, lower alkynyl, phenyl, cinnamyl or other ester groups. Optionally, the amino group can be protonated to isolate the product as an amine acid halide salt or the like.

In accordance with another preferred embodiment, there is provided a process that uses 4-nitrophenol to produce ethyl 2-(6-amino-chroman-2-yl)acetate 7 as set forth in the reaction Scheme XIX which follows on a later page.

Referring to Scheme XIX, the individual preferred reaction steps illustrated therein will now be discussed in more detail. In the first step, an embodiment provides a cyclization reaction wherein maleic acid and a phenol react under acidic conditions producing a 4-chromanone, as shown above in Scheme XVII. A preferred phenol is 4-nitrophenol to provide compound 3 as the product. To the yellow solid is added a mixture of H₂SO₄/EtOH, prefelably at room temperature, to form an ester 8. An organic solution containing the ester 8 is washed with water and brine, dried over magnesium sulfate, and concentrated under vacuum to yield the purified product. Typically, the overall yield of this process is about 85-95%.

An alternative embodiment to extend the chain and obtain a 2-chroman-2-yl acetic acid or ester thereof utilizes the Amdt-Eistert reaction. In the next steps, compound 2 may be converted first into an acyl halide 3, as shown in Scheme 1. Then, the acyl halide 3 is converted to a diazoketone 12 by nucleophilic attack of diazomethane on the acyl halide 3, as shown below.

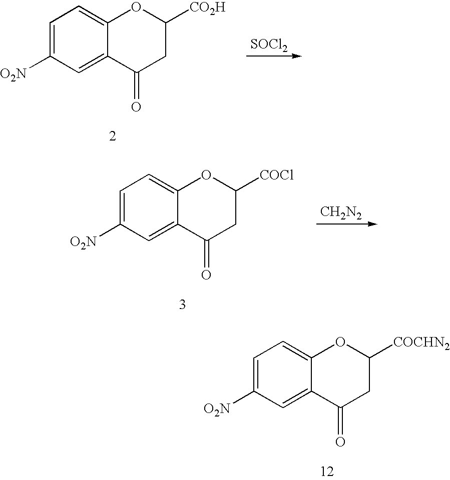

Then, the diazoketone can be converted into a carboxylic acid group with an additional methylene group from the original substrate in good yield by water and a catalyst such as Ag₂O, as follows:

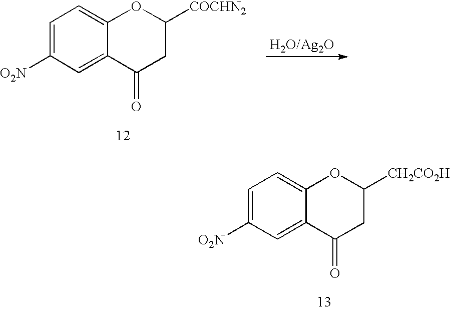

The process then proceeds by compound 13 undergoing hydrogenation to convert the nitro group into an amino group. Examples of conditions to reduce the nitro group include catalytic hydrogenation and use of chemical reducing agents in acidic solutions. A preferred condition for this conversion is the use of hydrogen in the presence of catalytic palladium/carbon and glacial acetic acid. Then, compound 6 is esterified in acidic ethanol to yield compound 7, as shown below.

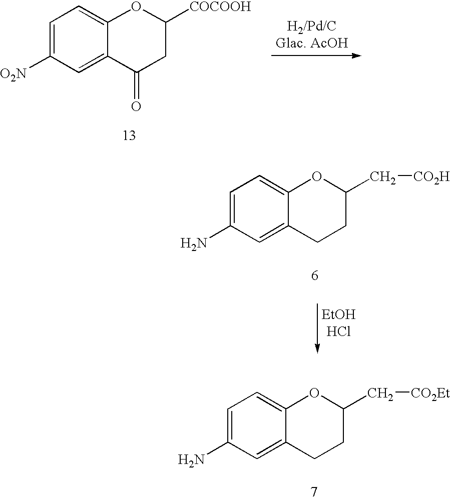

Preferably, R² is an ethyl group that results from acidic ethanol.

While the ethyl group was used to form the ester of the acetic acid side chain in the last step, the ethyl group can be replaced by hydrogen or another group capable of forming an ester selected from lower alkyl, lower alkenyl, lower alkynyl, phenyl, cinnamyl or other ester groups. Optionally, the amino group can be protonated to isolate the product as an amine acid halide salt or the like.

The above discussion is a general description of preferred processes. Non-limiting Illustrative Schemes 1 to 19, that are set forth below, show additional preferred embodiments.

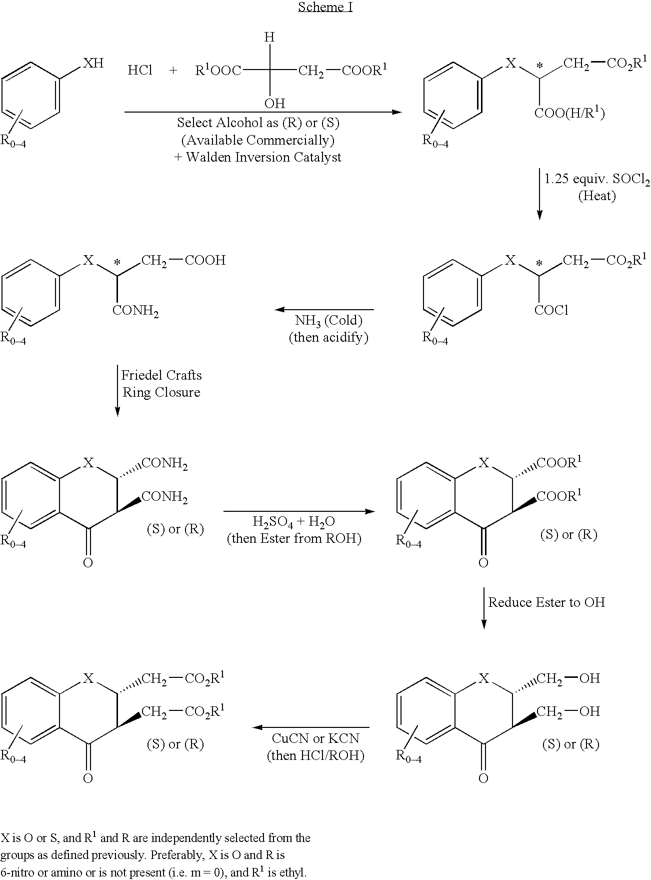

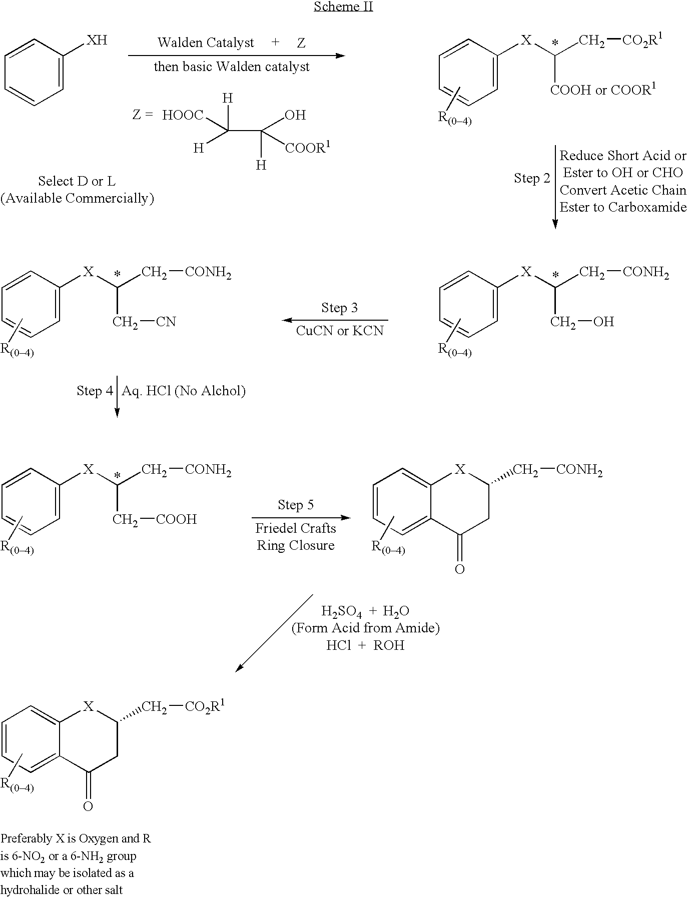

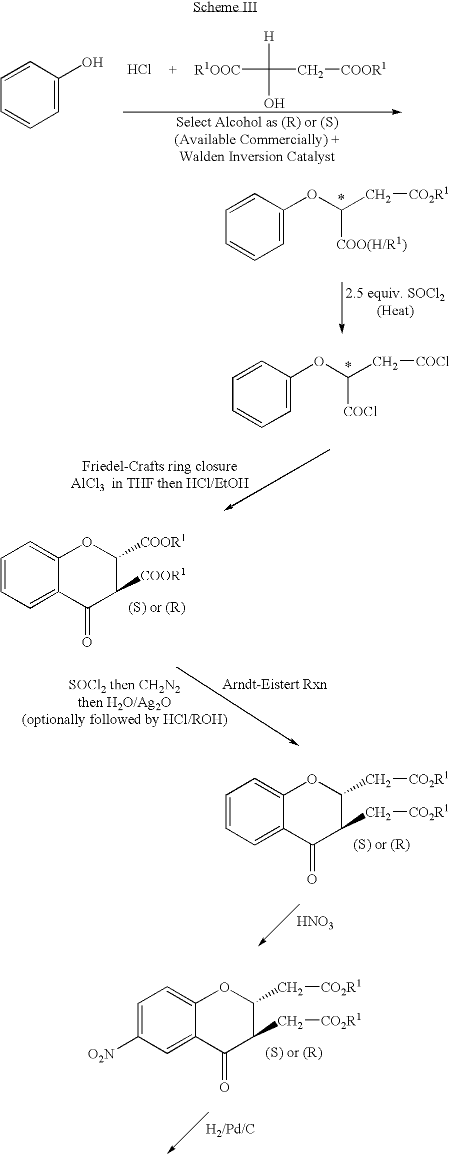

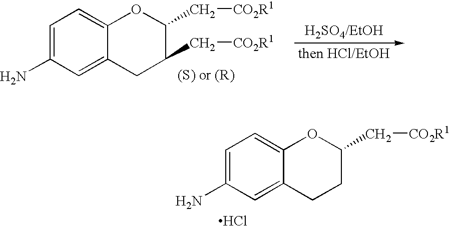

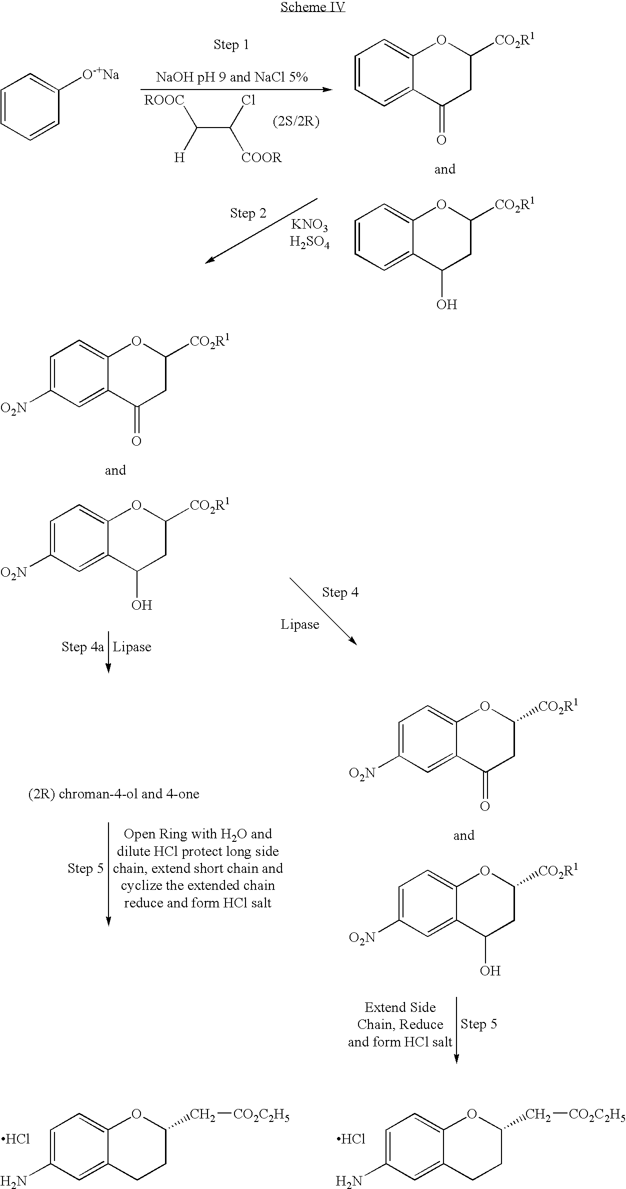

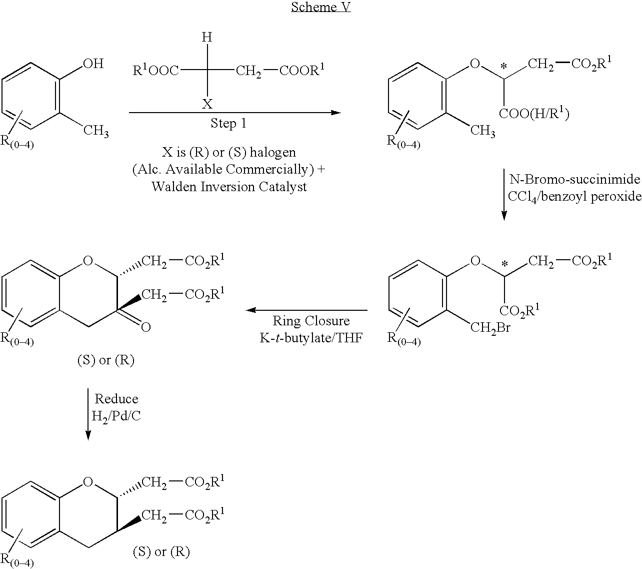

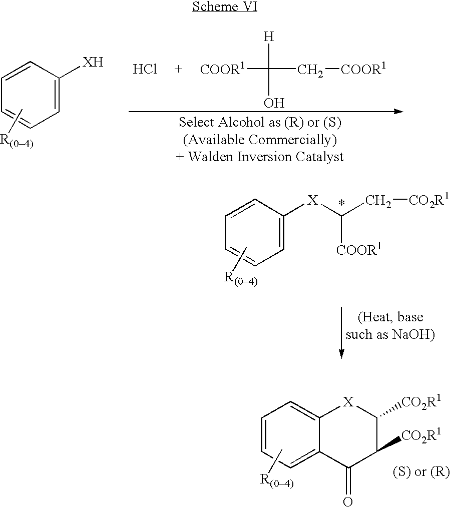

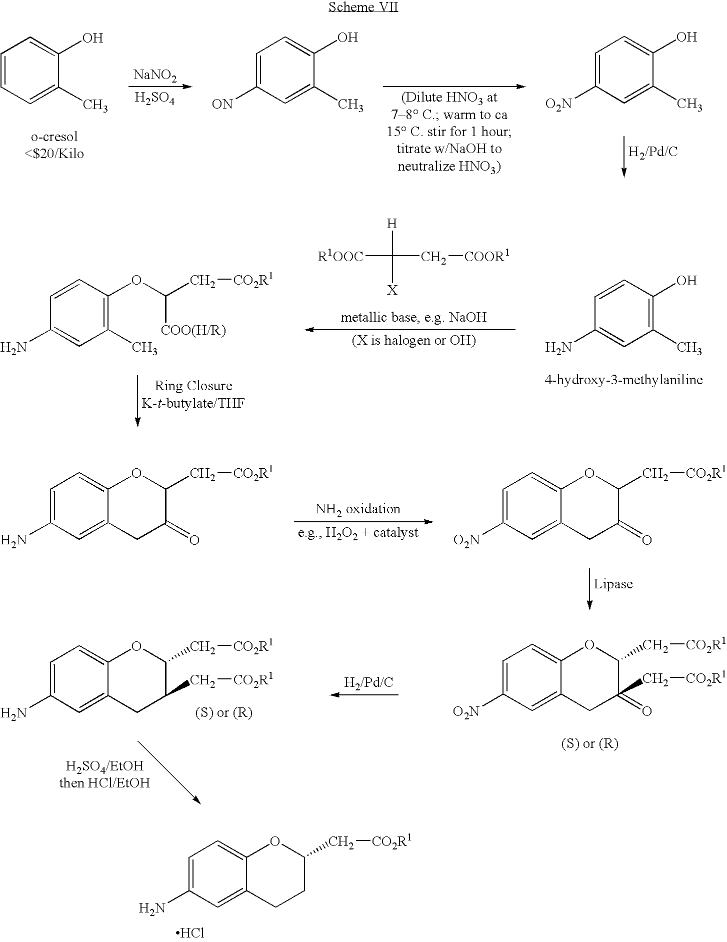

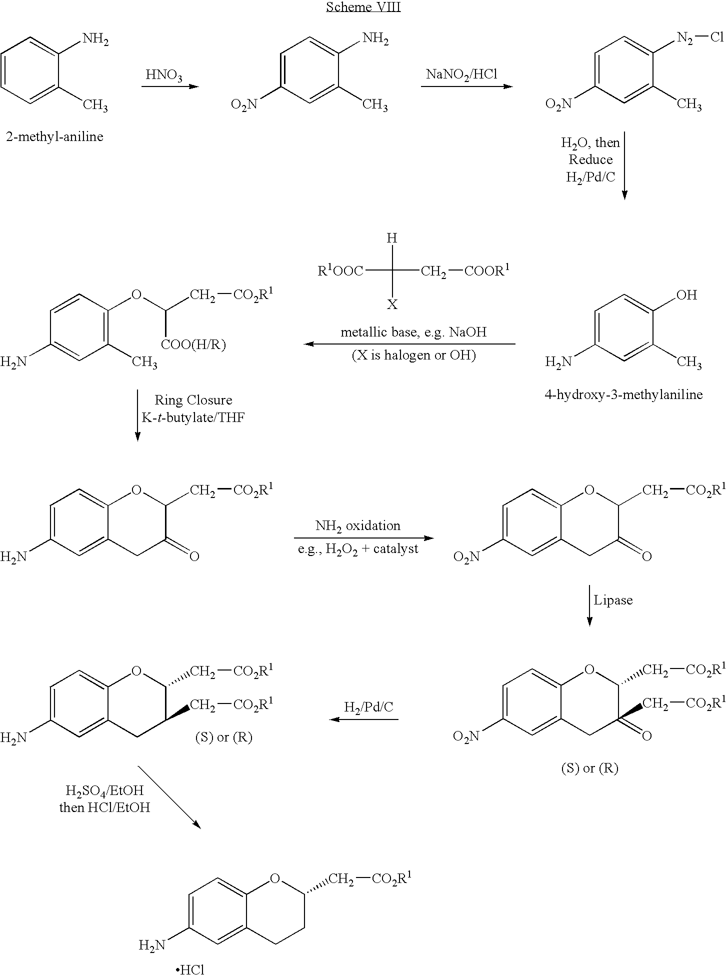

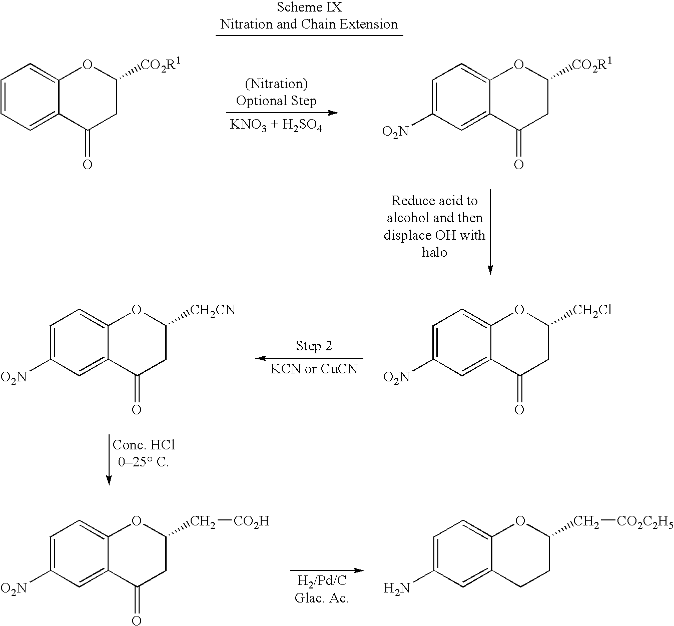

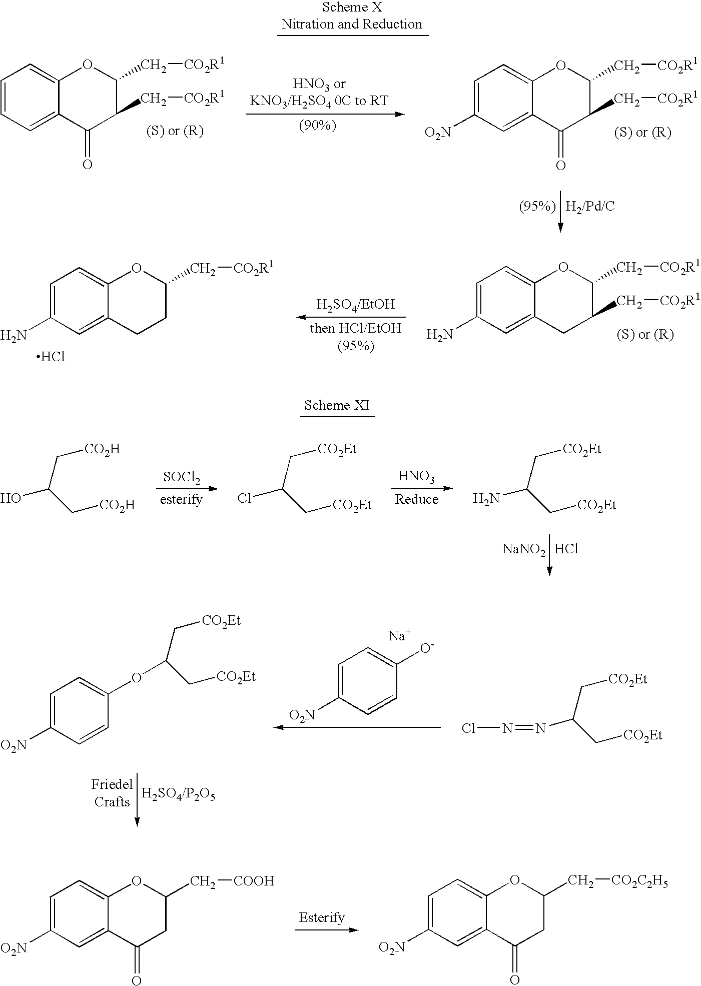

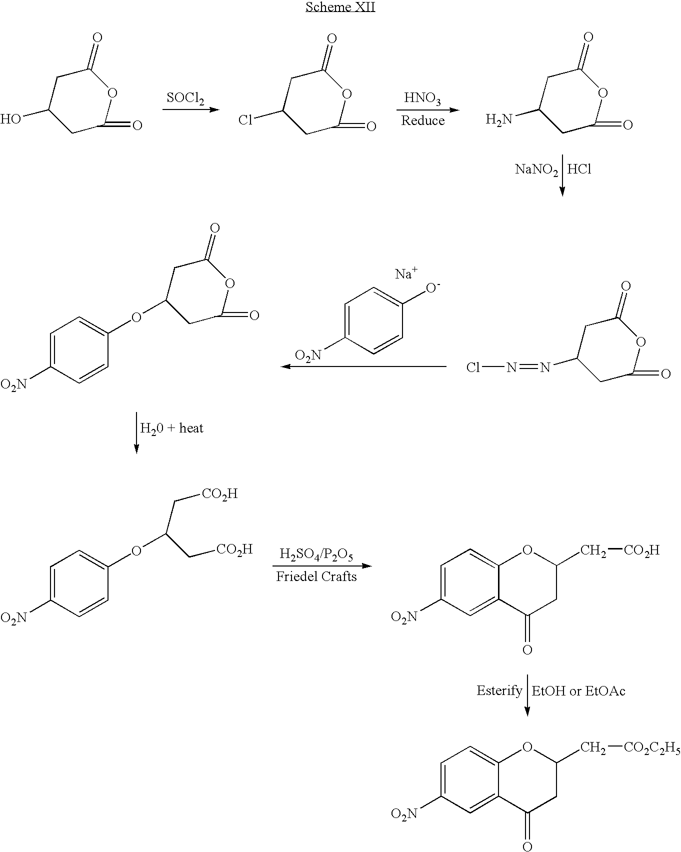

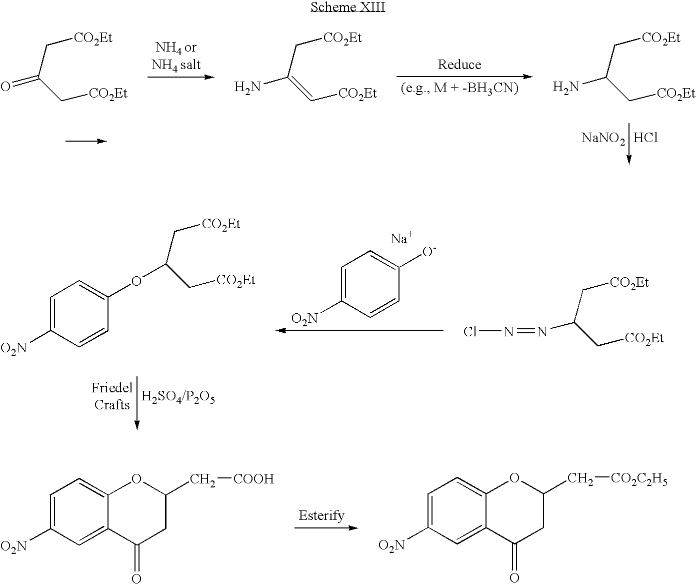

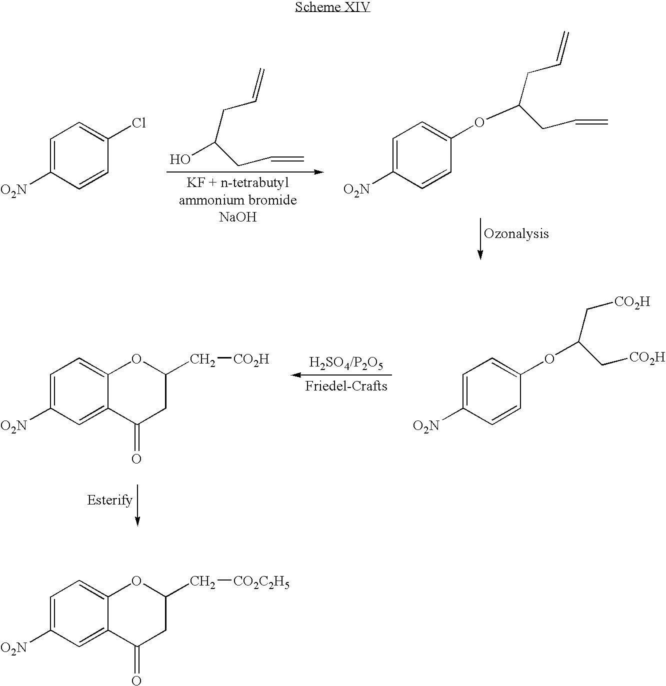

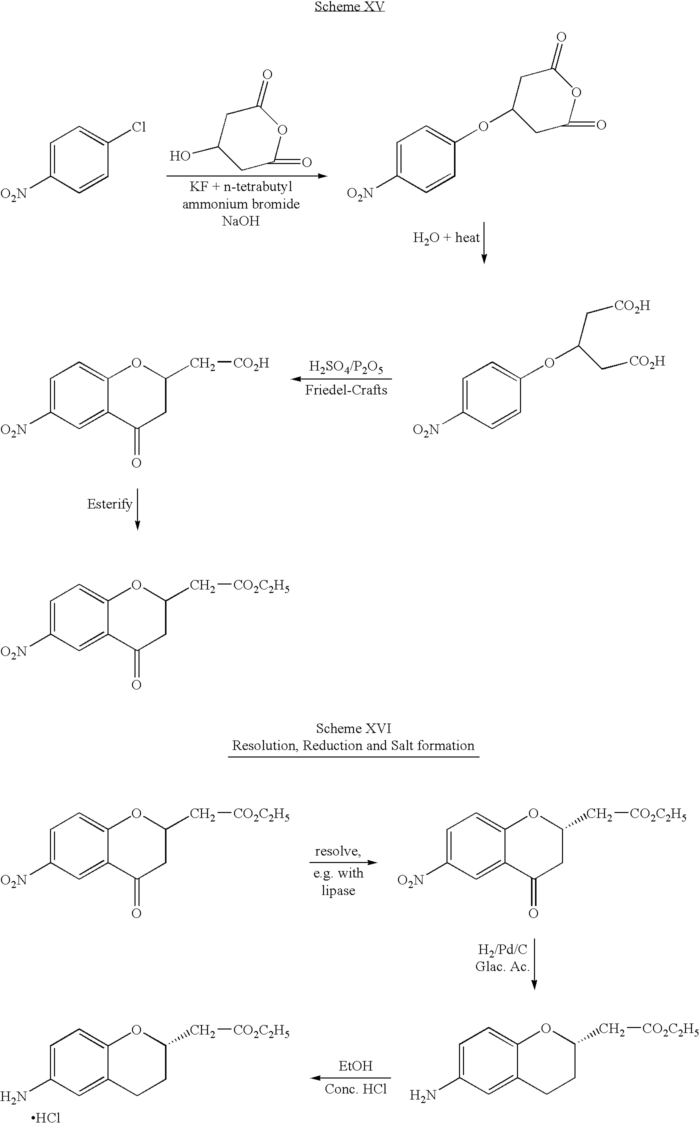

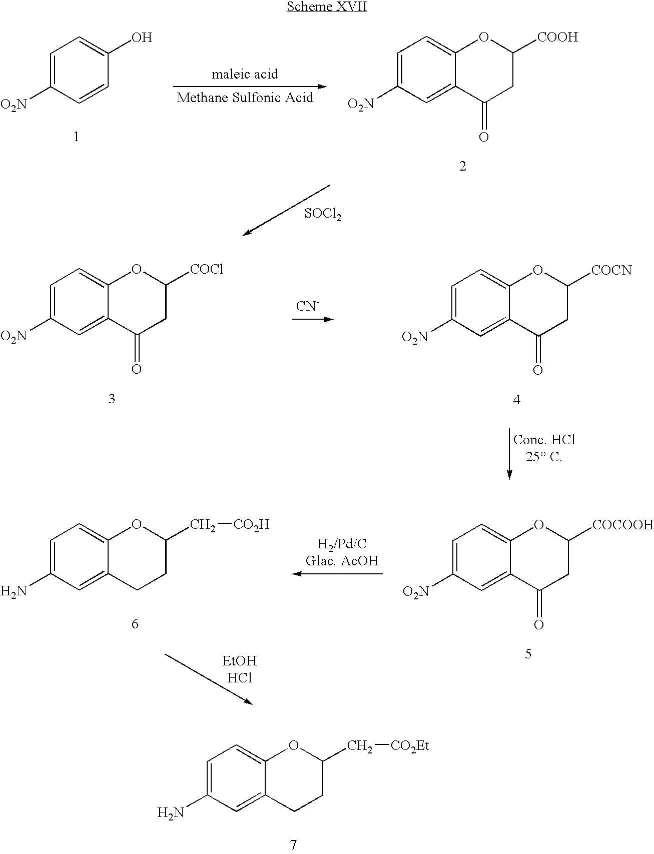

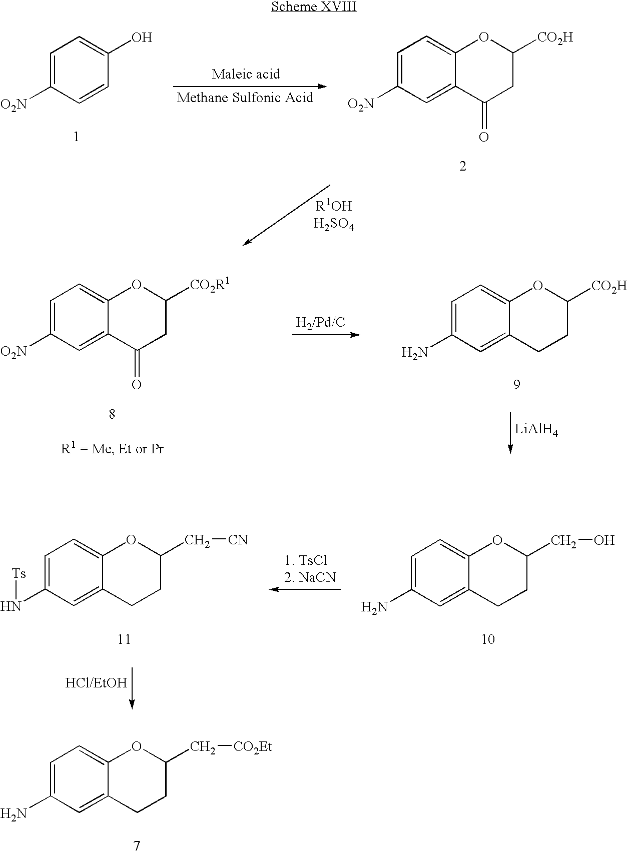

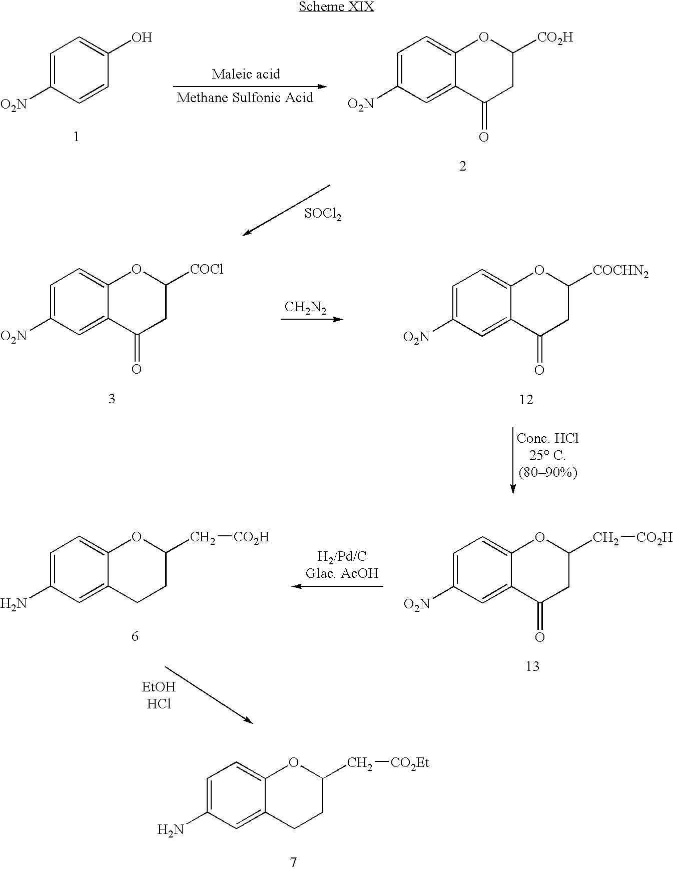

In Schemes I through VIII, although either a hydroxyl or halo malonate or succinate compound is shown in each scheme, a corresponding hydroxyl or halo substituted compound may be used in the reactions shown herein.

Schemes I and III each show a slightly different procedure to obtain the 4-oxochroman-2-l carboxylate ester depending upon whether 1.25 or 2.5 equivalents of thionyl chloride are added. These procedures may be substituted for each other such that the process having fewer steps is used along with the other steps in Scheme I and vice-versa.

In Scheme V and VII the ring closure (with or without bromination) can be replaced by formation of an acylhalide on the shorter chain (where R¹ is preferably H) by reaction with SO Cl₂ followed by ring closure using a strong base such as N-butyl lithium to remove one H from the ring methyl group to promote cyclization. Alternatively, procedures from preceding schemes can be utilized to perform a Friedel-Crafts type cyclization.

In Schemes VII and VIII, it is preferred that X is halide. The lipase resolution steps of Schemes VII and VIII may be omitted by using an (R) or (S) chiral halide to couple with the phenol in the manner of Schemes I, II, III, V, or VI. The nitroso compound of Scheme VII can be directly hydrogenated to the amine without formation of the nitro intermediate.

Schemes XI through XV all of which form ethyl 2-(4-oxo-6-nitrochroman-2-yl)acetate are preferably followed by Scheme XVI, or a variant thereof, in which the racemates are resolved, the reduced and/or a salt is formed. In another embodiment, the order of the steps of Scheme XV are changed such that resolution follows reduction, or that there is a first reduction of either the 4-oxo group or the 6-nitro group, followed by reduction of the other group and resolution of the racemate, which may proceed in either order. In some embodiments, salt formation may be omitted. However, the salt form is preferred in one embodiment of coupling reaction. Furthermore, the reactions may proceed with suitable reagents other than those shown in the Scheme, as are known in the art.

In the schemes shown above, the order of some of the reactions may be reversed, some (e.g. chain extension and nitration) some reactions may be omitted (e.g. a final esterification or salt formation step), and reactions from other schemes may be substituted in for other reations having similar results. Additionally, other reagents and conditions having similar results may be substituted for those disclosed. For reactions producing racemates, resolution of the racemate may occur at other suitable places in the scheme and may proceed with reagents other than lipase, a preferred reagent and process disclosed herein. Furthermore, salts and esters may be formed and/or interconverted with the corresponding free acid or base as desired at any place in the scheme if desired, such as to aid in isolation or purification of a compound or intermediate.

In other embodiments, the ordor of some of the reactions in the schemes may be changed, and additional steps of protecting, deprotecting, nitrating, hydrolyzing, esterifying, and the like may be added to the schemes at various points. Such minor alterations are within the scope of the disclosure herein. Although the esters shown are primarily ethyl esters, other esters may be made, either by use of different solvents and/or reagents in the initial formation reactions or by transesterification.

The starting materials used in the disclosed processes are commercially available from chemical vendors such as Aldrich, Lancaster, TCI, Bachem Biosciences, and the like, or may be readily synthesized by known procedures including those present in the chemical literature, or may be made by using procedures such as indicated above.

Reactions are carried out in standard laboratory glassware and reaction vessels under reaction conditions of standard temperature and pressure, except where it is otherwise indicated, or where use of non-STP conditions for a procedure is known in the art. Some procedures, reactions, and/or workups which are well known in the art or which are readily available in standard reference texts in the art, including Beilstein and Fieser and Fieser, may not be presented herein owing to their stature of being within the knowledge of one of ordinary skill. Further, the above procedures of the processes may be carried out on a commercial scale by utilizing reactors and standard scale-up equipment available in the art for producing large amounts of compounds in the commercial environment. Such equipment and scale-up procedures are known to the ordinary practitioner in the field of commercial chemical production.

During the synthesis of these compounds, amino or acid functional groups may be protected by blocking groups to prevent undesired reactions with the amino group during certain procedures. Procedures for such protection and removal of protecting groups are routine and well known to the ordinary practitioner in this field.

Enantiomeric Resolution and Acid Salt Formation

When a reaction results in the production of racemic chroman-2-yl carboxylic acids and esters or their derivatives and/or intermediates, these racemates are preferably resolved to produce a mixture enriched in one of the R or S enantiomers or resolved into a substantially pure composition of one of the enantiomers. Examples of processes for resolving the racemic mixtures are provided herein and others are known to those skilled in the art. Additionally, processes for the formation of acid addition salts such as the hydrochloride salt of the 6-position amino acid group on the chromane nucleus are known in the art. Other such salts are also envisioned.

As noted above, in preferred aspects, the methods disclosed herein relate to processes for producing amidino-substituted benzoyl compounds, wherein the phenyl ring may be substituted with lower alkyl, lower alkoxy, Cl, F, Br, I, and the like, which are intermediates for coupling with bicyclic compounds to produce therapeutic agents, or are themselves therapeutic agents, for disease states in mammals that have disorders caused by or impacted by platelet dependent narrowing of the blood supply. Some of such methods disclosed herein include processes for producing racemic amino substituted bicyclic compounds such as racemic, 6-amino-chroman-2-yl acetic acid esters, and resolving such bicyclic compounds into either the R or S enantiomer.

In a preferred aspect, racemic nitro substituted compounds having the bicyclic structures described below, are enzymatically resolved into an enantiomerically rich composition (2R>2S) or (2S>2R). Such resolution is preferably performed using a chirally selective Pseudomonas lipase such PS 30, or a stablized lipase (glutarate stablized, for example) such as the Altus, Inc. ChiroCLEC-PC lipase, or the like, may be utilized to resolve the nitro-substituted chromane, hydroxy chromane, or oxo-chromane compounds and the like.

One preferred resolution process (shown for a chromone) is as follows:

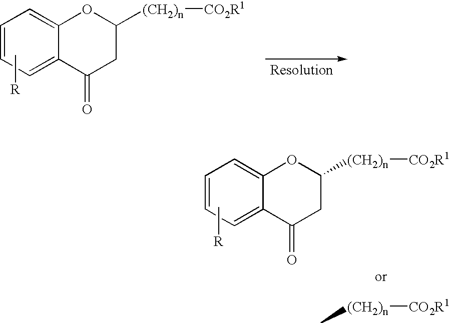

wherein R is a nitro or other electron withdrawing group on the phenyl or benzene ring such as an oxime or halogen group and R1 is hydrogen or the alkyl core of an alcohol group which can form an ester, such as a methylene or ethylene group from methanol or ethanol and n is 0 to 6. Although a chromone is shown, the process works on other rings as well. Preferably, the 2-carboxylic acid group is esterified with a methyl or ethyl group and the R group on the phenyl portion is a member selected from NO₂, halogen, an oxime derivative or the like. More preferably, R group is an 6-nitro group.

In a preferred aspect, PS 30 from Pseudomonas is used to selectively hydrolyze a 2-acyl ester group, or to catalyze selectively esterifying the free acid. When PS-30 is used, the Senantiomer is selectively hydrolyzed and in an aqueous basic/organic solvent can be extracted in the aqueous layer as a basic salt. The aqueous solution can then be neutralized to obtain the free acid. Alternatively, the organic portion of the aqueous/organic solvent extraction is enriched in the R-enantiomer which can then be recovered by heating in a base to form a basic salt that precipitates from the organic solvent. The yield is about 40-50% of 95% or greater purity of the desired crude enantiomer. Essentially 100% pure single enantiomer can be obtained from the 95% or greater crude enantiomer by refluxing the crude enantiomer in methanol. The amount of methanol solvent utilized in the desired purification reflux step can be varied to produce optimum yields of the desired pure enantiomer.

Where resolution of a 6-amino-chroman-2-yl acid (or an acid derivative) or other bicyclic structures that are substituted by an amino group is desired, although the process may proceed with the amino group, the process preferably first proceeds by oxidation of the amino group to a more electron withdrawing nitro group. Altematively, the reaction is begun with a nitro-substituted material. Preferably, the amino-compound is reacted with an oxygen source, including but not limited to, O₂, ozone, or H₂O₂, optionally in the presence of a catalyst such as a metal oxide catalyst selected from the group consisting of tungstate, molybdate and vanadate to result in oxidation of the amino group. In one preferred embodiment, an effective amount of hydrogen peroxide of from about 1-10, more preferably 1-5 moles of hydrogen peroxide per mole of amino groups is used in an aqueous or partially aqueous solution of a lower alcohol solvent to convert the ring amino group of the Formula I compound to a nitro group on the Formula II compound as follows:

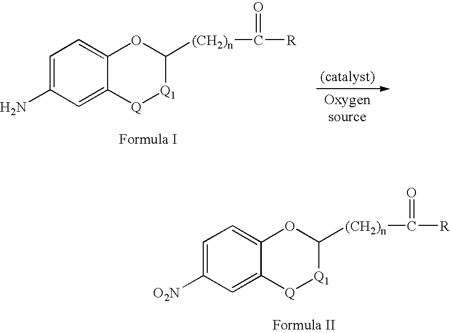

wherein in each of Formula I and Formula II n is 0 to 6; R is a member selected from the group consisting of alkoxy, alkenyloxy, halogen, amino, substituted amino and the like, and each of Q and Q₁ are independently selected from the —C(—R², —R³)—, wherein each of R² and R³ is independently selected from the group consisting of lower alkyl, lower alkoxy, lower alkenyl, hydroxy, thio, nitro, halo, and CF₃, or R¹ and R² collectively with the carbon to which they are attached form a carbonyl group.

In a preferred aspect, the oxidation of the amino to a nitro group is carried out at a temperature from about 20° C. to about 100° C., preferably from about 40° C. to about 85° C., more preferably from about 55° C. to about 75° C., and most preferably at about 60° C.

In a preferred aspect, the oxygenation catalyst for the amino to nitro conversion is a metal oxide catalyst selected from the group consisting of tungstate, molybdate and vanadate. Examples of tungstate or water soluble quaternary ammonium tungstates include tungstic acid (H₂WO₄), tungsten trioxide (WO₃), tungstenic acid (H₂WO₄), sodium tungstate (Na₂WO₄), potassium tungstate (K₂WO₄), and mixtures thereof. The corresponding molydbates, MoO₃, H₂MoO₄, K₂MoO₄, Na₂MoO₄, and mixtures thereof, and the corresponding vanadates Va₂O₅, HVO₃, KVO₃, NaVO₃, and mixtures thereof may be utilized. The catalyst is typically present at a level of from 0.001% to 2%, preferably 0.01% to 1% and more preferably from 0.05% to 0.5%, by weight of the aqueous or partially aqueous solution.

A chelating agent or heavy metal ion sequestrant such as organic phosphonates, EDTA (ethylene diamine tetra-acetic acid) and the like, may be utilized during the oxidation reaction, preferably diethylenetriamine penta(methylene phosphonate) (“DTPMP”). Such complexes are described in U.S. Pat. No. 4,259,200. The preferred concentration of sequestrant is approximately a 1:1 ratio to the metal catalyst ions utilized.

In a preferred aspect, the mixture is resolved by using an enantiomerically selective ester hydrolyzing agent such as a lipase, preferably a Pseudomonas lipase, most preferably PS 30 or a glutarate stablized version (for example ChiroCLEC-PC lipase for Altus, Inc.). In a preferred embodiment the selective hydrolysis by the lipase is conducted in an aqueous basic solution (preferably a buffer solution) with lipase PS 30. In this process, the insoluble ester racemate is agitated with stirring and the hydrolyzed acid forms a salt that is soluble in the aqueous solution. The solution can be filtered and the hydrolyzed acid (2S) can be recovered from the aqueous solution by neutralizing the solution to reform the water-insoluble free acid from the salt and thus recover the insoluble free acid as a precipitate. Rinsing this precipitate with water will yield the (2S) enantiomer free acid. The lipase biomass and the enriched (2R) enantiomer are preferably recovered by rinsing the biomass with an appropriate solvent such as ethyl acetate, filtering the ethyl acetate solvent and evaporating the solvent to recover the enriched (2R) enantiomer.

Depending upon whether the desired enantiomer is the (2S) or the (2R) enantiomer, the less desired enantiomer can be recycled by using a racemization step followed by exposure of the resulting racemate to the lipase to obtain more of the desired (2S) or (2R) enantomer and increase the overall yield of the process. The formation of a racemate from a single enantiomer is preferably accomplished by exposing the enantiomer to a basic alcoholic solution such as a sodium or potassium ethanolate solution in the corresponding alcohol or an inert solvent. Other procedures which open the ring at the ring oxygen of the chromane and then reclose it may also be utilized to produce a racemate from a single enantiomer. By repeating the resolution and racemate forming steps a higher overall yield may be obtained. The racemate forming step may be illustrated in a preferred compound as follows:

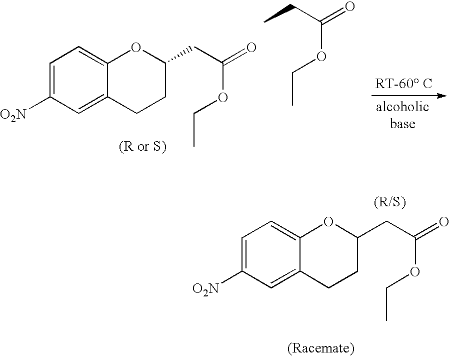

wherein, as illustrated, a catalytic amount of sodium ethoxide, potassium ethoxide or similar catalytic base in R¹OH (preferably EtOH) is utilized until racemization is completed, usually for 4-8 hours at about 45° C. (longer at room temperature). After acidification with an acid such as 1N HCl (preferably acetic acid) to quench the base and form a soluble salt with the base, the reaction mixture containing the racemic acid mixture is mixed with a greater volume of water than the volume of the alcohol solvent to render the ethyl ester of the racemic (2R/2S) 6-nitro-chroman-2-yl acetic acid insoluble. The racemic mixture is collected as a precipitate by filtration and is rinsed with water. Optionally, the crude product can be thoroughly rinsed with water and recystallized in an appropriate solvent to ensure that the sodium or potassium ions are removed from the racemate. The resulting ester racemate can then be recycled by exposure to the lipase to obtain a higher yield of the desired single enantiomer with respect to the initial amount of racemate starting material.

The separated (2S>2R) or (2R>2S) enantiomer from the optional chiral resolution step (for illustration purposes only the 2S>2R enantiomer is shown below) may undergo further reactions as set forth herein, below or above. Such reactions include, but are not limited to chain lengthening, nitration, salt formation, esterification, hydrolysis, and reduction of oxo, nitro and other groups. For example, a compound having the 6-nitro group is hydrogenated to yield back the amino of Formula I as an enriched enantiomer which can be utilized as in intermediate for coupling to a benzoyl compound as described in U.S. Pat. No. 5,731,324 to provide a specific enantiomerically enriched platelet aggregation inhibitor compound. The hydrogenation process is demonstrated below as follows with the (2S>2R) 6-nitro-chroman-2-yl acetic acid compound, but any bicyclic nitro containing compound of Formula II may be thus hydrogenated back to the Formula I structure after the resolution as follows:

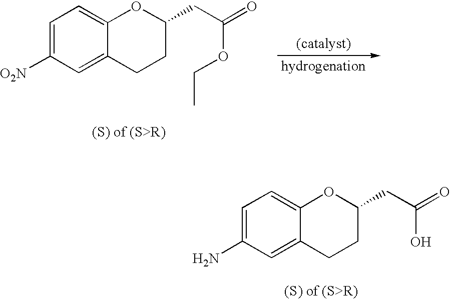

After the hydrogenation is completed, the compound can be re-esterified and an amine salt (preferably a hydrohalide) may be formed to precipitate the ester out of what is preferably an organic solvent solution. For example, an alcoholic sulfuric acid solution followed by an alcoholic HCl solution can be utilized for the esterification and forming hydrohalide salt of the amino group, but other esters including the methyl or propyl esters may likewise be used. Salts other than HCl may also be used. The purity of the enantiomer may be optionally improved by recrystallation, HPLC or the like. The preferred solvent for recrystallization is methanol or isopropyl alcohol or a mixture thereof. In a preferred embodiment, after resolving the desired (2S or 2R)-enantiomer and obtaining the 2-carboxylic acid form of the molecule, the acidic alcoholic solutions may be added to the free acid R or S enantiomer compound to form a 2-carboxylic acid ethyl ester (or another ester) and an excess of HCl is then utilized to produce the hydrochloride salt of the amino group, as follows

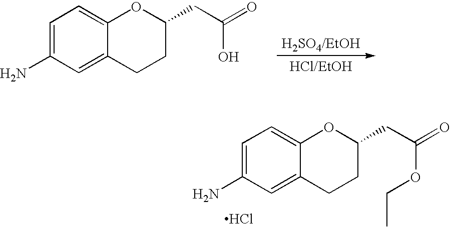

While an ethyl group was used to form the ester of the acetic acid side chain, the ethyl group can be replaced by H or another esterfing group selected from lower alkyl, lower alkenyl, lower alkynyl, phenyl, cinnamyl or other ester groups.

Uses of Compounds

As mentioned above, the compounds produced according to preferred embodiments find utility as intermediates for producing therapeutic agents or as therapeutic agents for disease states in mammals, including those which have disorders that are due to platelet dependent narrowing of the blood vessels, such as atherosclerosis and arteriosclerosis, acute myocardial infarction, chronic stable angina, unstable angina, transient ischemic attacks and strokes, peripheral vascular disease, arterial thrombosis, preeclampsia, embolism, restenosis following angioplasty, carotid endarterectomy, anastomosis of vascular grafts, and etc. These conditions represent a variety of disorders thought to be initiated by platelet activation on vessel walls.

Platelet adhesion and aggregation is believed to be an important part of thrombus formation. This activity is mediated by a number of platelet adhesive glycoproteins. The binding sites for fibrinogen, fibronectin and other clotting factors have been located on the platelet membrane glycoprotein complex IIb/IIIa. When a platelet is activated by an agonist such as thrombin, the GPIIb/IIIa binding site becomes available to fibrinogen, eventually resulting in platelet aggregation and clot formation. Thus, intermediate compounds for producing compounds that effective in the inhibition of platelet aggregation and reduction of the incidence of clot formation are useful intermediate compounds.

The compounds produced according to preferred embodiments may also be used as intermediates to form compounds that may be administered in combination or concert with other therapeutic or diagnostic agents. In certain preferred embodiments using the compounds disclosed herein and/or the compounds formed from coupling such compounds with substituted benzoyl halides and/or other compounds may be co-administered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds produced from the intermediates may act in a synergistic fashion to prevent reocclusion following a successful thrombolytic therapy and/or reduce the time to reperfusion. Such compounds may also allow for reduced doses of the thrombolytic agents to be used and therefore minimize potential hemorrhagic side-effects. Such compounds can be utilized in vivo, ordinarily in mammals such as primates, (e.g. humans), sheep, horses, cattle, pigs, dogs, cats, rats and mice, or in vitro.

Coupling Reaction of the Hydrochloride Salt Intermediate Compounds

The above compounds produced according to preferred methods may be isolated and further reacted to substitute a desired group for one or more of the hydrogen atoms on the amino group by a coupling reaction. Particularly preferred is a coupling reaction of the amino group with an acyl halide compound. For example, compounds such as 5-amidino-thiophen-2-yl carboxylic acid derivatives (or an acyl halide such as the acyl chloride) and 4-amidinobenzoyl chloride may be coupled to ethyl 2-[(2S) 6-aminochroman-2-yl] acetate (or its hydrochloride salt) to form ethyl 2-[(2S) 6-(5-amidino-2-thiophenoyl)amino-chroman-2-yl]acetate and ethyl 2-{(2S)-6-[(4-amidinophenyl) carbonylamino]chroman-2-yl} acetate, or other similar compounds or their derivatives which are known platelet aggregation inhibitors. For examples of such platelet aggregation inhibitors, see U.S. Pat. No. 5,731,324. The ring portion of the above amidino-aroyl or amidino-heteroaroyl derivatives may be substituted by groups such as methyl, ethyl, fluoro, iodo, bromo, chloro, methoxy, ethoxy, and the like which results in compounds that are known platelet aggregation inhibitors. Standard coupling procedures may be utilized, but procedures utilizing reaction mixtures the compounds, in salt form, are suspended in solvents such as acetonitrile, toluene, or the like, are preferred.

The compound formed from the coupling reaction may be used as either the salt or the free base, and may be readily interconverted between the two forms by using procedures which include those known in the art as well as reacting the compound with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product may be passed over an ion exchange resin to form the desired salt, or one salt form of the product may be converted to another using the same general process. The free base or salts may be purified by various techniques such as recrystallization in a lower alkanol such as methanol, ethanol, propanol, isopropanol and the like, for example, or a mixture thereof. In preferred embodiments, the compound is recovered as the hydrochloride salt and the recrystallization solvent is a 90/10-10/90 mixture of ethanol and isopropanol. Non-toxic and physiologically compatible salts are preferred, although other types of salts may also be used, such as in the processes of isolation and purification.

Compositions and Formulations

Diagnostic and therapeutic applications of the compounds formed by procedures disclosed herein, including the aforementioned coupling reactions, will typically utilize formulations wherein the compound, or a pharmaceutically acceptable salt, solvate, or prodrug, is combined with one or more adjuvants, excipients, solvents, or carriers. The formulations may exist in forms including, but not limited to tablets, capsules or elixirs for oral administration; suppositories; sterile solutions or suspensions for injectable or parenteral administration; or incorporated into shaped articles. Subjects in need of treatment (typically mammalian) using the compounds disclosed herein and/or the compounds formed from coupling such compounds with substituted benzoyl halides and/or other compounds can be administered dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize.

Formulations are prepared for storage or administration by mixing the compound, or a pharmaceutically acceptable salt, solvate or prodrug thereof, having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counter ions such as sodium and/or nonionic surfactants such as Tween, Pluronics or polyethyleneglycol.

Dosage formulations to be used for parenteral administration are preferably sterile. Sterility is readily accomplished by filtration through sterile membranes such as 0.2 micron membranes, or by other conventional methods known to those skilled in the art. Formulations are preferably stored in lyophilized form or as an aqueous solution. The pH of such preparations are preferably between 3 and 11, more preferably from 5 to 9 and most preferably from 7 to 8. It will be understood that use of certain of the foregoing excipients, carriers, or stabilizers will result in the formation of cyclic polypeptide salts. While the preferred route of administration is by injection, other methods of administration are also anticipated such as intravenously (bolus and/or infusion), subcutaneously, intramuscularly, colonically, rectally, nasally or intraperitoneally, employing a variety of dosage forms such as suppositories, implanted pellets or small cylinders, aerosols, oral dosage formulations and topical formulations such as ointments, drops and dermal patches. The compounds are desirably incorporated into shaped articles such as implants which may employ inert materials such as biodegradable polymers or synthetic silicones, for example, Silastic, silicone rubber or other polymers commercially available.

The compounds may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compounds may also be delivered by the use of antibodies, antibody fragments, growth factors, hormones, or other targeting moieties, to which the compound molecules are coupled. The compounds may also be coupled with suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the platelet aggregation inhibitors may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

Therapeutic compound liquid formulations generally are placed into a container having a sterile access port, for example, an intravenous solution bag or vial having a stopper pierceable by hypodermic injection needle.

Therapeutically effective dosages may be determined by either in vitro or in vivo methods. For each particular compound and formulation, individual determinations may be made to determine the optimal dosage required. The range of therapeutically effective dosages will naturally be influenced by the route of administration, the therapeutic objectives, and the condition of the patient. For injection by hypodermic needle, it may be assumed the dosage is delivered into the body's fluids. For other routes of administration, the absorption efficiency must be individually determined for each inhibitor by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be within the ambit of one skilled in the art. Typically, applications of compound are commenced at lower dosage levels, with dosage levels being increased until the desired effect is achieved.

A typical dosage might range from about 0.001 mg/kg to about 1000 mg/kg, preferably from about 0.01 mg/kg to about 100 mg/kg, and more preferably from about 0.10 mg/kg to about 20 mg/kg. Advantageously, the compounds or formulations may be administered several times daily, in a once daily dose, or in other dosage regimens.

Typically, about 0.5 to 500 mg of a compound or mixture of compounds, as the free acid or base form or as a pharmaceutically acceptable salt or prodrug derivative (including esters), is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

Typical adjuvants which may be incorporated into tablets, capsules and the like are a binder such as acacia, corn starch or gelatin, and excipient such as microcrystalline cellulose, a disintegrating agent like corn starch or alginic acid, a lubricant such as magnesium stearate, a sweetening agent such as sucrose or lactose, or a flavoring agent. When a dosage form is a capsule, in addition to the above materials it may also contain a liquid carrier such as water, saline, a fatty oil. Other materials of various types may be used as coatings or as modifiers of the physical form of the dosage unit. Sterile compositions for injection can be formulated according to conventional pharmaceutical practice. For example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

The compounds and formulations may be used alone or in combination, or in combination with other therapeutic or diagnostic agents. In certain preferred embodiments, the compounds and/or formulations may be coadministered along with other compounds typically prescribed for these conditions according to generally accepted medical practice, such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds and formulations can be utilized in vivo, ordinarily in mammals such as primates, such as humans, sheep, horses, cattle, pigs, dogs, cats, rats and mice, or in vitro.

The compounds, selected and used as disclosed herein, are believed to be useful for preventing or treating a condition characterized by undesired thrombosis, such as (a) the treatment or prevention of any thrombotically mediated acute coronary syndrome including myocardial infarction, unstable angina, refractory angina, occlusive coronary thrombus occurring-post-thrombolytic therapy or post-coronary angioplasty, (b) the treatment or prevention of any thrombotically mediated cerebrovascular syndrome including embolic stroke, thrombotic stroke or transient ischemic attacks, (c) the treatment or prevention of any thrombotic syndrome occurring in the venous system including deep venous thrombosis or pulmonary embolus occurring either spontaneously or in the setting of malignancy, surgery or trauma, (d) the treatment or prevention of any coagulopathy including disseminated intravascular coagulation (including the setting of septic shock or other infection, surgery, pregnancy, trauma or malignancy and whether associated with multi-organ failure or not), thrombotic thrombocytopenic purpura, thromboanginitis obliterans, or thrombotic disease associated with heparin induced thrombocytopenia, (e) the treatment or prevention of thrombotic complications associated with extracorporeal circulation (e.g. renal dialysis, cardiopulmonary bypass or other oxygenation procedure, plasmapheresis), (f) the treatment or prevention of thrombotic complications associated with instrumentation (e.g. cardiac or other intravascular catheterization, intra-aortic balloon pump, coronary stent or cardiac valve), and (g) those involved with the fitting of prosthetic devices.

Without further description, it is believed that one of ordinary skill in the art can, using the preceding description and the following illustrative examples, make and utilize the compounds disclosed herein and practice the claimed methods. The following working examples therefore, specifically point out preferred embodiments, and are not to be construed as limiting in any way the remainder of the disclosure.

### EXAMPLES

### Example 1

Production of diethyl 2-(4-nitrophenoxy)butane-1,4-dioate

A mixture containing 78.5 g of 4-nitrophenol and 194.0 g of the diethyl ester of 3-bromomalic acid was heated to 120° C., 75.1 g of potassium carbonate were added slowly and the resulting mixture was then heated at 110° C. for 5 hours. The temperature was lowered to 100° C. and the reaction was then diluted with 1.5 liters of aqueous ethanol and then acidified to pH 1, at 80° C. with concentrated sulfuric acid. After concentration of the solvent under vacuum, the residue was taken up with water and ethyl ether. Gel formation was observed. After decantation, the organic phase was washed with a 4% solution of sodium hydroxide and then with water. It was dried over sodium sulfate and then the solvent was concentrated under vacuum to provide about 210 g of an oil diethyl (2-(4-nitrophenoxy)butane-1,4-dioate, m.w 285.54), a yield of approximately 70-75%.

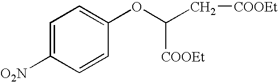

### Example 2

Production of 2-(2-nitrophenoxy)-butan-1,4-acyl chloride

To a 3-neck round bottom flask equipped with a mechanical stirrer, nitrogen inlet, reflux condenser, heating mantle, vacuum system, and scrubber system for efficient removal of HCl and SO₂ gas which was liberated during the reaction, was charged under nitrogen 0.5 moles of thionyl chloride, and 0.2 moles of the diethyl (2-(4-nitrophenoxy)butane-1,4-dioate obtained from Example 1, above. The stirred mixtures was placed under a N₂ flow, which was vented to the scrubber system. The stirred mixture was heated to reflux for 12 hours during which the acylation reaction becomes complete. The resulting solution was placed under vacuum and excess thionyl chloride was removed by evaporation under vacuum. The resulting product, below, was obtained in about 95% yield.

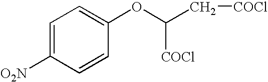

### Example 3

Production of 6-nitro-4-oxo-chroman-2-yl carboxylic acid ethyl ester (Ftiedel-Crafts Ring Closure Reaction)

The mixture obtained from Example 2 and 300 mL of anhydrous tetrahydrofuran were charged under nitrogen into a 1 L 3-neck round bottom flask equipped with a mechanical stirrer, nitrogen inlet, 500 mL addition funnel, cooling system, and thermowell. The reaction mixture was stirred and cooled to about −60° C., 1.5 moles of AlC₃ were added through the addition funnel and the funnel was rinsed with 25 mL of anhydrous tetrahydrofuran. The solution was stirred for about an hour at −75° C. The mixture was gradually warmed to room temperature while maintaining stirring, and the reaction was quenched with 80 mL of HCl. Then 50 mL of concentrated HCl and 100 mL of EtOH were added to the reaction mixture to produce 38 g of the 6-nitro-4-oxo-chroman-2-yl carboxylic acid ethyl ester product, shown below (about 75% yield).

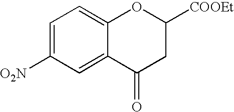

### Example 4

Production of diethyl 2-(4-nitrophenoxy)butane-1,4-dioate

An alternate method for making the above captioned compound with respect Example 1 was as follows: A mixture containing 80 g of 4-nitrophenol, 45 g of potassium hydroxide, 200 g of the diethyl ester of 3-bromomalic acid, 750 mL of ethanol and 7.5 mL of Aliquot 336®, a quaternary ammonium compound marked by Aldrich Fine Chemicals, was refluxed for 8 hours. The solvent was concentrated and the residue was taken up with 1.5 L of H₂O. Extraction was carried out with ethyl ether and the organic phase was washed with a 1 N solution of sodium hydroxide and then with water. It was dried over sodium sulfate and concentrated. The product was optionally purified by chromatography on a silica column using pentane as the eluent. About 100 g of diethyl 2-(4-nitrophenoxy)butane-1,4-dioate was obtained (about 60% yield).

### Example 5

Production of 1-(2-(4-nitrophenoxy)-4-ethyl butanoate) acyl chloride

A 3-neck round bottom flask equipped with a mechanical stirrer, nitrogen inlet, reflux condenser, heating mantle, vacuum system, and scrubber system for efficient removal of HCl and SO₂ gases liberated during the reaction, was charged under nitrogen with 0.25 moles of thionyl chloride, and 0.2 moles of the diethyl 2-(4-nitrophenoxy)butane-1,4-dioate obtained from Examples 1 or 4 above. The stirred mixture was placed under a N₂ flow, which was vented to the scrubber system. The stirred mixture was heated to reflux for 12 hours during which the reaction became complete. The resulting solution was placed under vacuum and excess thionyl chloride was removed by evaporation under vacuum. The resulting product 1-(2-(4-nitrophenoxy)-4-ethyl butanoate) acyl chloride (below) was obtained in about 95% yield.

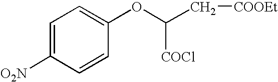

### Example 6

Production of 1-(2-(4-nitrophenoxy)-4-ethyl butanoate) carboxamide

A 3-neck round bottom flask equipped with a mechanical stirrer, nitrogen inlet, reflux condenser, heating mantle, vacuum system, and scrubber system for efficient removal of HCl and SO₂ gases liberated during the reaction, was charged under nitrogen with 0.1 moles of thionyl chloride, and 0.2 moles of the diethyl 2-(4-nitrophenoxy)butane-1,4-dioate obtained from Examples 1 or 4, above. The stirred mixture was placed under a N₂ flow, which was vented to the scrubber system. The stirred mixture was heated to reflux for 12 hours during which the reaction became complete. The resulting solution was placed under vacuum and excess thionyl chloride was removed by evaporation under vacuum. The resulting product 4-(1-(4-nitrophenoxy)-4-ethyl butanoate)-1-acyl chloride was reacted with NH₃ under standard carboxamide forming conditions and the resulting product 1-(2-(4-nitrophenoxy)-4-ethyl butanoate) carboxamide, below, was obtained in about 95% yield.

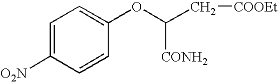

### Example 7

Production of 6-nitro-4-oxo-chroman-2-yl carboxamide (Friedel-Crafts Ring Closure Reaction)

Using the carboxamide product of Example 6, the procedures of Example 3, above, were followed except that the final step of adding EtOH and concentrated HCl was eliminated. Such procedures yield about 40 g of the 6-nitro-4-oxo-chroman-2-yl carboxamide product (about 75% yield).

### Example 8

Production of 6nitro-4-oxo-chroman-2-yl carboxylic acid ethyl ester

The carboxamide product of Example 7 was added to concentrated H₂SO₄ in 300 mL of EtOH under standard ester forming conditions to yield the ethyl ester. The yield was about 40 g of the 6-nitro-4-oxo-chroman-2-yl carboxylic acid ethyl ester product (about 95% yield).

### Example 9

Production of (S) Diethyl Malonate

In a mixture of 500 mL of distilled water and 200 mL of EtOH was added 134 g (1 mole) of L-malic acid (S) with stirring, followed by 10 mL of 1N H₂SO₄. The mixture was heated to 70° C. for 1 hour with continuous stirring and then removed from the heat. The mixture was allowed to cool to room temperature and 10 mL of 1N NaOH was added to neutralize the H₂SO₄ of the mixture, which was then extracted with 4×100 mL of acetone. The organic layers were combined and the solvent was evaporated under vacuum to yield about 190 g of the diester product, (S) diethyl malonate (about 1 mole).

### Example 10

Production of (R) diethyl 2-chlorosuccinate

The (S) diethyl malonate of Example 9 (1 mole) was added to 250 mL of anhydrous EtOH and 1 mole of concentrated HCl (essentially anhydrous) was added to the mixture with stirring. The temperature was raised to 60° C. with stirring and was maintained at this temperature for up to 3 hours with continuous strring(until HPLC indicates that all of the alcohol had been converted to its halide derivative). The solution was cooled to 0° C. and filtered to remove inorganic salts. The EtOH solvent was then evaporated off under vacuum to yield about 208.5 g of crude (R) diethyl 2-chlorosuccinate (1 mole). The solid was rinsed with cold (0° C.) distilled water to remove inorganic materials. Optionally the diester was re-suspended in EtOH for combination with a metallic oxide to produce an ether derivative.

### Example 11

Production of (S) diethyl 2-chlorosuccinate

The (S) diethyl malonate of Example 9 (1 mole) was added to 250 mL of anhydrous EtOH, and 1 equivalent of SOCl₂ (essentially anhydrous) was added to the mixture with stirring. The temperature was raised to 60° C. with stirring, and was maintained at that temperature for up to 3 hours with continuous stirring (until HPLC indicated that all of the alcohol had been converted to its halide derivative). The solution was cooled to 0° C. and filtered to remove inorganic salts. The EtOH solvent was then evaporated under vacuum to yield about 208.5 g of crude (S) diethyl 2-chlorosuccinate (1 mole). The solid was rinsed with cold (0° C.) distilled water to remove inorganic materials. Optionally the diester was re-suspended in EtOH for combination with a metallic oxide to produce an ether derivative.

### Example 12

Production of an Alkaline Aqueous Sodium Phenoxide Solution

60 grams of NaOH were slowly added to 500 mL of distilled water, and the mixture was heated to 100° C. with stirring. About 140 g of phenol crystals were slowly added to the hot alkaline solution with stirring to avoid excessive boiling as the resulting hydrogen gas was released. The temperature was increased to 130° C. and the pH was adjusted to 9.0. The mixture was maintained at this temperature for 30 minutes and 10 g. of NaCl was added with stirring to yield a hot alkaline aqueous sodium phenoxide solution in brine containing about 1.5 moles of sodium phenoxide.

Coupling and Cyclization Reaction Steps

### Example 13

Production of (2S) Diethyl Succinate Phenyl Ether

The (2R) diethyl 2-chlorosuccinate of Example 10 (1 mole) in 250 mL of EtOH was slowly added (dropwise) at 120° C. with stirring to the alkaline aqueous sodium phenoxide solution at a pH of 9, which was maintained by adding NaOH to the mixture. After the diester and EtOH had been added to the alkaline sodium phenoxide solution, the temperature was raised to 135° C. and maintained with stirring with the occasional addition of 25 mL of EtOH until HPLC indicates that all of the halide had been consumed and converted to its ether derivative by coupling with the phenoxide. The solution was cooled to 100° C. and acetic acid was added to consume the NaOH until a pH of 8 was obtained. The aqueous mixture was extracted with 4×150 mL of acetone and the organic layers were combined and extracted with 2×100 mL of a dilute aqueous NaOH solution to remove the excess phenol. The pH of the organic layer was adjusted to 7.0 with acetic acid and the solvent was evaporated under vacuum to yield a reddish-orange oil which includes the (2S) diethyl succinate phenyl ether intermediate (below).

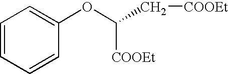

### Example 14

Production of ethyl chroman-4-one 2-carboxylate

The (2S) diethyl succinate phenyl ether of Example 13 in 250 mL of EtOH was added in small portions to an aqueous NaOH solution at 135° C. while maintaining a pH of 9.0. The mixture was stirred continuously and the temperature was raised to 145° C. while the pH was maintained at 9.0 by alternatively adding 1N NaOH or concentrated acetic acid. The mixture was stirred at this temperature and 25 mL of EtOH was added every 15 minutes until HPLC indicated that all of the ether had been cyclized to yield the chromanone. The solution was cooled to 100° C. and acetic acid was added to consume the NaOH until a pH of 7.0 was obtained. The aqueous mixture was extracted with 4×150 mL of ethyl acetate and the organic layers were combined. The organic layer was extracted once with 100 mL of distilled water and the organic layer was concentrated to an orange/red solid or oil by evaporation under vacuum, which includes the crude (2S) ethyl chroman4-one 2-carboxylate (or its corresponding (2S) 4-hydroxy derivative).

### Example 15

Production of (2R) Diethyl Succinate Phenyl Ether

The (S) diethyl 2-chlorosuccinate of Example 11 (1 mole) in 250 mL of EtOH was utilized in the process as set forth in Example 5 to yield a reddish-orange oil which includes the (2R) diethyl succinate phenyl ether intermediate.

### Example 16

Production of ethyl chroman-4-one 2-carboxylate

The (2R) diethyl succinate phenyl ether of Example 15 in 250 mL of EtOH was utilized in the process as set forth in Example 14 to yield an orange/red solid or oil by evaporation under vacuum, which includes the crude (2R) ethyl chromantone 2-carboxylate (or its corresponding (2S) 4-hydroxy derivative).

### Examples 17-24

Preparation of Thio-Derivatives

Examples 9-16 were repeated with the corresponding thiophenol starting materials and the corresponding thioether and bicyclic thio derivatives were obtained in substantially the same yields.

### Example 25

Preparation of 2-hydroxy-5-nitroso-toluene

Into a 2-liter round bottomed flask (sifting on ice) o-cresol (34.5 g, 0.319 mole), ammonium nitrite (NH₄NO₂, M.W. ˜60, 0.393 mole) in 80 mL of H₂O (29.2% by weight solution of NH₄NO₂ in water), and conc. sodium hydroxide (0.755 mole) in 500 mL of water were combined, and ice water was added to adjust the overall volume of the mixture to 700 mL while stirring the mixture. The NaOH was used to cause the o-cresol to be more soluble in water and to cause the resulting 2-hydroxy-5-nitroso-toluene to be soluble in the water so long as it remains as an aqueous alkaline solution. With continued stirring, the mixture was cooled to 2° C. and maintained at that temperature for one hour with stirring. After one hour, a total of 0.765 moles of H₂SO₄ as an aqueous solution (0.765 moles dissolved in 240 mL of water) which had been chilled to 0° C. was gradually added with stirring at such a rate as to maintain the temperature of the resulting mixture below 50° C.

After the H₂SO₄ had all been added, the reaction mixture was filtered cold and the filter cake was washed three times with 250 mL of ice water. Drying of the filter cake yields 30.2 g of >98% pure 2-hydroxy-5-nitroso-toluene (M.W. 137.14).

If the 5-nitroso compound was to be converted to a 5-nitro compound by oxidation without further ring substitution, a higher yield of the nitro compound can be obtained by changing the washing steps to merely rinsing the filter cake once with 25 mL of ice water. The resulting crude nitroso compound can be used as the reactant for the oxidation step and the resulting nitro compound which was insoluble in water can be separated from any residual o-cresol by washing it with water after the oxidation step.

### Example 26

Preparation of 2-hydroxy-5-nitro-toluene

The crude filter cake obtained in Example 25, above, was added to 250 mL of H₂O, 50 mL of toluene and 150 g of ammonium molybdate in a 2 liter round bottom flask. The mixture was heated to reflux (about 100° C.) and maintained at reflux while a 500 mL aliquot of 3% by weight aqueous solution of hydrogen peroxide was added. Alternatively, 50 mL of a 30% by weight solution may be added. After the hydrogen peroxide had been added, the mixture was maintained at reflux for 4 hours. The solution was removed from heat and filtered while hot and the supernatant was set aside. The filter cake was washed three times with 100 mL of hot water, dried and set aside. The organic layer was shaken three times with 100 mL of hot water and the organic layer was concentrated under vacuum to dryness to yield a small amount of the product which was added to the earlier obtained filter cake. The cake was then dried under vacuum to obtain 34 g of 2-hydroxy-5-nitro-toluene (approximately 70% yield) in about 98% purity.

### Example 27

Preparation of 2-methyl-4 amino-phenol

The crude filter cake obtained in Example 26, above is placed in a hydrogenator, and the 4-position nitroso group (or nitro group) was reduced to an amine group with H₂/Pd/C in a hydrogenator with EtOH and toluene as the solvent. The reaction was monitored by HPLC until essentially 100% conversion was obtained. The resulting phenolic amine and solvent were removed from the hydrogenator, placed in a one liter 3 neck RB flask, and heated to about 60° C. with stirring. To the stirring mixture was added a sufficient amount of aqueous 1N NaOH or sodium carbonate to raise the pH above 8. HPLC monitoring was used to indicate conversion of substantially all of the phenol to the phenoxide and then the organic solvent was removed under reflux with vacuum. After removing the organic solvent, a sufficient amount of 1N HCl was added to the reaction mixture adjust the pH of the basic solution to about 9 and provide an aqueous basic solution of 2-methyl-4-aminophenoxide. (about 85-90% yield).

### Example 28

Preparation of diethyl 3-aminoglutarate

A room temperature solution of 40 g of ammonium acetate in 400 mL of ethanol is adjusted to a pH of about 6.0 by the addition of acetic acid. To this solution is added 10 g of diethyl 3-keto-glutarate. The mixture is stirred at room temperature for 1½ hours.

To this reaction solution is added 2.5 g of sodium cyanoborohydride (NaBH₃CN) slowly in small portions over about 5 hours. The reaction is stirred for 22 hours at ambient temperature and the pH is then adjusted to about 5.5 by the addition of acetic acid. After adjusting the pH, 0.65 g of sodium cyanoborohydride is added. The reaction mixture is stirred at room temperature for about 9 hours.

The reaction mixture is concentrated to less than 100 mL under reduced pressure at a temperature below 30° C., and the concentrated solution is adjusted to a pH of about 9.0 by the addition of aqueous saturated potassium carbonate and then extracted twice with 200 ML portions of CH₂Cl₂. The aqueous phase is adjusted to a pH of about 10 by adding an aqueous saturated potassium carbonate solution and is extracted twice with 200 mL of methylene chloride. The organic extracts are combined, washed once with a saturated solution of sodium chloride in water and dried over anhydrous sodium sulfate to yield about 9 g of a crude oily diethyl 3-aminoglutarate product.

### Example 29

Preparation of diethyl 3-diazenylpentane-1,5-dioate

To a reaction vessel cooled to about −10° C. is added successively anhydrous HCl (0.8 mole), ammonium chloride (50 mmoles) and the 9 g of crude oil product of Example 28 (about 50 mmoles). Diazotization is accomplished by the addition of sodium nitrite (53 mmoles) at about 0° C. with stirring. Anhydrous toluene that is cooled to the temperature of the reaction mixture is added to the reaction mixture and the organic and inorganic layers are separated to provide the diazotized reaction product in the organic layer.

### Example 30

Preparation of diethyl-3-(4-nitrophenoxy)pentane-1,5sdioate

To a reaction vessel cooled to about −0° C. is added the organic layer of Example 29. To this mixture is slowly added 50 mmoles of sodium nitrophenoxide with stirring. After addition of the sodium nitrophenoxide the reaction mixture is allowed to slowly come to room temperature and 50 mmoles of pyridine is added with stirring. The reaction mixture is stirred at room temperature for 6 hours and the organic solvents are removed under reduced pressure. The off-white cake is diluted with 100 mL of ice water and stirred for 1-2 hours at ice bath temperature. The mixture is stored cold overnight and filtered cold to yield about 20 g of an off-white powder.

Alternatively, the procedures of Examples 29 and 30 can be performed using 4-amino-3H-4,5-dihydropyran-2,6-dione as the material which is diazotized and reacted with nitrophenoxide. The resulting product is heated in the presence of water to obtain diethyl 3-(4-nitrophenoxy)pentane-1,5-dioate, the product of Example 30.

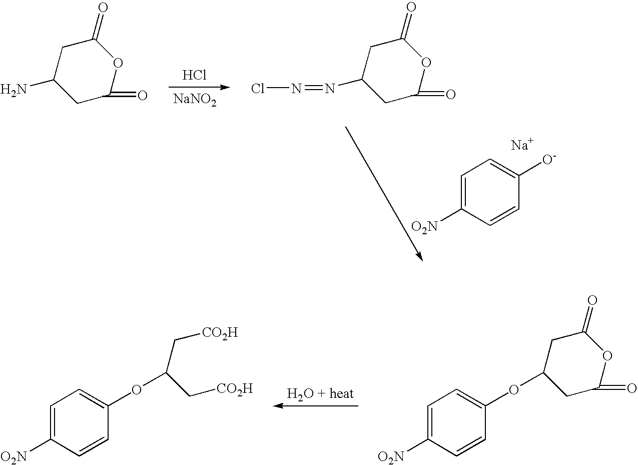

### Example 31

Preparation of ethyl 2-(4-oxo-6nitrochroman-2-yl)acetate

The 20 g of product from Example 30 is added to a reaction vessel and 80 g of concentrated sulfuric acid (cooled to about 5-15° C.) is slowly added to reaction vessel. While maintaining this temperature by cooling to 5 to 15° C. in 5 portion is slowly added 20 g of phosphorous pentoxide. The resulting solution (pale orange/brown) is stirred at about 10° C. for about 30 minutes and then allowed to slowly want to room temperature (10 to 15 minutes). The reaction mixture is quenched by slowly pouring the reaction mixture over 300 g of ice and 125 mL of water and stirred for 1-2 hours at ice bath temperature. A yellow gum is formed which slowly turns to a yellow tan powder. The yellow tan powder is dissolved 200 mL of ethyl acetate and 100 mL of water. The organic layer is separated and the aqueous layer extracted with two 100 mL portions of ethyl acetate. The organic layers are combined and the organic solvent removed under reduced pressure. The off-white cake is washed with two 50 mL portions of cold water. The cake is added to 150 mL of water and shaken for 1 hour, stored cold overnight and filtered cold to yield an off-white powder. The off-white powder is dried under vacuum to yield about 17 g of 6-nitrochroman-4-one acetic acid ethyl ester.

### Example 32

Production of 4-(4-nitrophenoxy)-1,6heptadiene

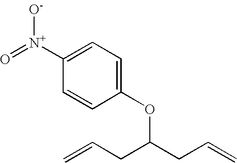

To a stirred solution of p-chloronitrobenzene (28 g, 0.178 mole) and 4-hydroxy-1,6-heptadiene (2 g, 0.1865 mole) in DMSO (170 mL) was added anhydrous KF (90.39 g., 6.9 mmoles) and n-tetrabutyl ammonium bromide (92.8 g, 8.7 mmoles) at ambient temperature. To the resulting pale yellow solution was added NaOH flakes (10 g, 0.25 mole) all at once. The yellow solution turned into a black solution in 2-3 minutes. The mixture was heated to 50° C. and stirred at that temp for 3.5 hours. The reaction was monitored periodically by GC. When the chloronitrobenzene was completely consumed (3.5 hours) the deep black suspension was diluted with tert-butyl methyl ether (600 mL) and stirred with 10% aqueous ammonium chloride 9150 mL) with ice cooling. The black 2-phase suspension was treated with decolorizing carbon (2 g) and filtered through a pad of celite.

The organic layer was separated, the aqueous layer was extracted with ether (2×100 mL) and then the combined organic layers washed with water (3×40 mL), dried over MgSO₄, filtered and evaporated to give 30 g of black gum. This gum was applied on a bed of silica gel (450 g) and eluted with 1.5 L of hexane. The resulting yellow filtrate on evaporation gave about 10.5 g of yellow oil which solidified on storage in the freezer. Further elution of the silica gel with 20% ether-hexane (1 L) gave on evaporation 4.3 g of impure 4-(p-nitro-phenoxy)-1,6-heptadiene as a yellow brown oil with a 9 to 1 mixture of the desired product and the diene-alcohol. The yellow-brown oil was then filtered through a pad of silica gel (60 g) with 200 ml of neat hexane. The filtrate was evaporated to give 3.6 gram of pure product as a thick yellow oil. Total isolated yield was 14.2 g (34%).

¹H NMR (CDCl₃): * 8.25 (2H, d), 7.05 (2H, d), 5.85 (m, 2H), 5.15 (m, 4H), 4.45 (m, 1 H), 2.5 (4H, d of d) ¹³C NMR (CDCl₃): * 163.39, 141.34, 133.05, 126.02, 118,47, 115.48, 76.02, 37.66

### Example 33

Production of 3-(4-nitrophenoxy)pentanedioic acid

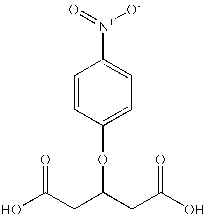

1.6 g of the compound of Example 32 (0.0326 moles), 150 mL of methylene chloride, and 15 mL of acetic acid were added to a 250 mL glass vessel with a 2 inch diameter glass frit at the bottom. The yellow solution was cooled to below −50° C. using a dry ice/acetone bath. The reaction was then sparged with approximately 2% ozoie in air at a rate of 2 liters per minute. After about an hour, the color changed to a greenish brown, and the ozone generator was turned off, allowing air to purge the excess ozone from the reaction (color faded back to yellow). The cooling bath was removed and the solution brought to room temperature.

The above mixture was left at ambient temperature overnight and the solution transferred to a 500 mL single neck R.B. flask and the solvents evaporated at 40-50° C. using a Buchi evaporator. All of the methylene chloride and some of the acetic acid was removed at this stage. The resulting yellow oil was diluted with 75 mL of glacial acetic acid and stirred while excess 50% aqueous hydrogen peroxide (4 g, 20 eq) was added in one portion and the mixture heated under mild reflux (110° C.) for 18 hours. At the end of this period HPLC showed 74% TAN of the compound 3-(4-nitrophenoxy)pentanedioic acid, with two major impurities and no trace of the starting material (from Example 1).

The reaction mixture was cooled to ambient temperature and then cooled with ice water whereby off-white powder deposited slowly during 2-3 hours. The precipitate was filtered off, washed with 4-5 mL hexane/acetone (1:1) and air dried. Further drying in the vacuum oven at 45-50° C. for 16 hours gave 2 g of off-white powder with LC purity of 96%. The mother liquor was evaporated to a yellow pasty powder. Ice water was added (20 ML) and the precipitated powder was filtered, washed with water 92 mL) and 2 ML of acetone/hexane 91:1) and vacuum dried in the oven at 45-50° C. for 16 hours to get another 1.5 g of off-white powder with LC purity of 95%. Total isolated yield (46%).

¹H NMR (CDCl₃): * 8.15 (2H, d), 7.15 (2H, d), 5.35 (m, 1H), 2.78 (4H, d). ¹³C NMR (CDC(₃): * 171.56, 162.59, 141.34, 125.59, 115.71, 72.02, 40.67

### Example 34

Production of 2-(6-nitro-4-oxo-chroman-2-yl) acetic acid

To a stirred solution of the compound of Example 33 (0.3 g, 0.0012 mole) in 1.25 g of concentrated sulfuric acid was added 0.24 g of phosphorous pentoxide slowly at room temperature. The resulting pale brown gummy oil was stirred at room temperate for 2 hours and then slowly poured into ice (5 g) and stirred for 10 minutes. The resulting yellow tan suspension was filtered, the filter cake washed with water (5 mL) and air dried. The tan powder was dried in the vacuum oven for 12 hours at 50° C. to get 0.24 g (78%) of a tan powder with an HPLC (TAN) purity of 96%.

¹H NMR (CDCl₃): * 8.85 (1H, s), 8.35 (1H, d), 7.35 (1H, d), 4.85 (m, 1H), 2.85 (2H, m) and 2.71 (m, 2H). ¹³C NMR (CDCl₃): * 189.57, 171.04, 165.15, 142.12, 130.27, 123.35, 120.26, 119.47, 75.29, 41.81 and 39.16

The following four examples, Examples 35-38, provide one preferred procedure for resolving racemic mixtures. In the examples, a racemic mixture of ethyl 2-(6-nitrochroman-2-yl) acetate is resolved. Other similar compounds having different substitutions on the ring may also be resolved by a process similar to that exemplified below.

### Example 35

Production of ethyl 2-(6nitrohroman-2-yl) acetate

To a 20 L RB three necked flask was added 2L of ethanol and 2 L of 30% w/w hydrogen peroxide solution with stirring followed by 280 mmoles of sodium tungstate, 1.4 moles of DTPMP solution (26% active) and 1.08 K g (4 mole) of ethyl 2-(6-amino-chroman-2-yl)acetate hydrochloride. The pH was adjusted with 1N HCl and NaOH to a pH of about 7.0 and the mixture was heated with stirring to about 65° C. to obtain a uniform suspension. The mixture was held there for 4-6 hours with stirring, until monitoring indicated complete conversion of the amino compound to the nitro derivative. The mixture was cooled and poured into 5 L of toluene. The organic layer was separated from the aqueous layer and the aqueous layer was extracted twice with 2 L of toluene. The organic layers and extracted organic portions were combined to provide an alcohol/toluene solution.

The alcohol/toluene solution of the nitro compound was made basic with sodium carbonate to precipitate the compound as a sodium salt. After filtration, the solid was added to 2 liters of water and sufficient 1N HCl was added to precipitate crude 6-nitro-chroman-2-yl acetic acid from the aqueous solution. The acid is esterified in absolute ethanol and the solvent is evaporated to obtain ethyl 2-(6-amino-chroman-2-yl)acetate in quantitative yield.

### Example 36

Enzymatic Resolution of Racemic ethyl 2-(6-nitrochroman-2-yl)acetate with a Lipase and Isolation of (R>S) and (S>R) Enantiomer Compositions.

A 50 L reactor was charged with 8 L of tetrahydrofuran, and 1 Kg (4.0 moles) of the wet racemic ethyl 2-(6-amino-chroman-2-yl)acetate (obtained as in Example 35) and 9 L of distilled water were sequentially added. With the aid of a pH controller, the pH of the stirring mixture was adjusted to 8 with a 3.0 N aqueous NaOH solution and the temperature was set to about 37° C. To this 37° C. mixture was added 20 g of a PS 30 lipase (PS 30 from Pseudomonas cepacia, Amano Enzyme Co., LTD, 1157 North Main Street, Lombard, Ill. 60148). The pH controller was set up to maintain a pH of about 8 and the peristaltic pump was set to maintain the pH by adding up to 750 mL of 3N sodium hydroxide solution. The container of NaOH only had 750 mL (2 moles) of 3.0 N sodium hydroxide (corresponds to roughly 50% of the moles of racemate) and this amount of NaOH was injected with in 24 hours, which indicates that at least 50% of the ester had been hydrolyzed and formed a sodium salt.

The THF was removed by rotoevaporation, the off-white suspension was adjusted to a pH of about 8 with NaOH and was then filtered through a vacuum filter funnel and carefully rinsed with 1 L of distilled water. The slightly basic aqueous filtrates containing the enriched (2S) enantiomer were combined and kept. The drain of the funnel containing the enriched (2R) enantiomer and solid bio-mass was sealed and 1 L of ethyl acetate was added to it. The ester and some free acid were dissolved and the solution was allowed to pass through the drain and collected. The insoluble bio-mass was further rinsed with 500 mL of ethyl acetate and the combined ethyl acetate washes were dried over 50 mg of sodium sulfate. After filtration and solvent removal by rotary evaporation, about 52% yield of the ethyl 2-((2R>S) 6-nitro-chroman-2-yl)acetate or free acid is collected.

The basic filtrates containing the enriched (2S>2R) enantiomer were transferred into a 20 L 3-necked RB flask, with the aid of a pH controller, the pH was carefully adjusted to about 3.5 by a slow addition of 8.2 L of 3 N hydrochloric acid. The formed white suspension was collected by vacuum filtration and thoroughly rinsed with 30.5 L of distilled water. The product was dried in a vacuum oven at 60° C. to afford about (40% yield) of 2-(2S>2R)-6-nitro-chroman-2-yl acetic acid (2S >90%).

### Example 37

Production of ethyl 2-(2S>2R)6amino-chroman-2-yl)acetate hydrochloride salt

Ten percent palladium on carbon (70 g of wet material; about 50% moisture) and 3 angstrom molecular sieves (100 g) were placed in a Parr hydrogenator. The bomb was purged 3 times with nitrogen. Anhydrous ethanol (2 L Kg) was added under vacuum while maintaining the reaction medium at room temperature, and 400 g of (2S>2R) 2-(6-nitro-chroman-2-yl) acetic acid as from Example 36 was added to the stirred solution. The bomb was purged 3 times with nitrogen and then 3 times with hydrogen, pressurized to 70 psi hydrogen, and heated to 80° C. while stirring. The reaction was monitored by TLC to indicate when the nitro groups had been hydrogenated, after which the reaction mixture was cooled to room temperature. The mixture was emptied from the bomb, and the bomb was rinsed with anhydrous ethanol which was added to the mixture, both of which were filtered through a celite bed. The catalyst and sieves were washed one time with 200 mL of ethanol. The filtrate and wash were combined and placed in a 20 L RB flask to which a catalytic amount of HCl was added. This mixture was heated to form the ethyl ester. The reaction mixture was refluxed and reduced to 1L, and 2 L of toluene was added. Concentrated HCl was added to precipitate the hydrochloride amine salt. The crystalline salt was filtered, washed twice with 100 mL of toluene and dried for about 16 hours under reduced pressure (20° C. <T <45° C.) to yield about 85%, ethyl 2-(2S>2R)6-amino-chroman-2-yl)acetate hydrochloride.

### Example 38

Optional Racemization of Ethyl (2R>2S) (6-nitro-chroman-2-yl)acetate for Recycling to Increase Yield of (2S>2R) Enantiomer

A 3 L 3-necked RB flask equipped with a heating mantle, condenser, overhead mechanical stirrer and thermocouple was charged with 1 L of ethanol, 500 g of the enriched ethyl 2-(2R>2S)6-amino-chroman-2-yl)acetate (having some free acid) obtained in Example 36 and 4 mL (100 mmoles) of sodium ethoxide, 21% w/w in ethanol. The mixture was heated to 45° C. and became a clear solution (the pH of the reaction was checked and adjusted to at least 10, by adding additional sodium ethoxide). The mixture was stirred at 45° C. for 6 hours. Analysis of the reaction by rotation was utilized to indicate complete reaction (an increase of about 3 degrees of rotation). To the mixture was then added 8 mL of acetic acid and the mixture was allowed to cool to room temperature before the mixture was added to 1 L of distilled water and shaken. The precipitate that was formed was filtered into a vacuum funnel. The combined portions of the filtered precipitate were rinsed with 500 mL of distilled water to yield the racemic ethyl (6-nitro-chroman-2-yl)acetate as a solid. Essentially 100% of the starting material appeared to be conserved and the racemate was collected wet for recycling through the Example 36 enzyme resolution procedure.

### Example 39

Production of 6-nitrochroman-4-one-2-carboxylic acid

To 4-nitrophenol (1 mol) was added maleic acid (2 mol) and 1120 mL of methanesulfonic acid, and mixture was heated to 92° C. for 20 h. After cooling to 0° C., the reaction mixture was poured onto ice water (2L) and extracted with diethyl ether (3×800 mL). The organic layers were combined; washed with water (3×500 mL), 1 N NaOH (4×500 mL), water (2×500 mL), and then brine (500 mL); dried over magnesium sulfate; and concentrated in vacuo to afford 6-nitrochroman-4-one-2-carboxylic acid (about 50 g, 40-45% yield) as a yellow solid.

### Example 40

Production of ethyl 6-nitrochroman-4-one-2-carboxylate

A solution of 6-nitrochroman-4-one-2-carboxylic acid (4.5 g) (Example 39) in concentrated hydrochloric acid (75 mL) was stirred vigorously at room temperature for about 6 h. After adding ethanol (150 mL) to the solution, the solution was allowed to stand overnight. The precipitate was filtered, rinsed with ether (150-mL), and dried. The precipitate was stirred with absolute ethanol (75 mL) and HCl (10 mL) for 2 h. An additional 10 mL of concentrated HCl was then added. The precipitate was collected and recrystallized twice from ether/isopropanol affording ethyl 6-nitrochroman4-one-2-carboxylate (about 4.4 g, 90% yield).

### Example 41

Production of ethyl 6acetamidochroman-2-carboxylate

A hydrogenation apparatus was charged with ethyl 6-nitrochroman-4-one-2-carboxylate (6 g) (Example 40), acetic anhydride (3.5 mL), 10% palladium on carbon (1 g), powdered 3 Å molecular sieves (4.0 g), and glacial acetic acid (30 mL). After purging several times with nitrogen, the apparatus was purged several times with hydrogen. Under continuous stirring, the apparatus was maintained at about 70 psi with hydrogen and about 80° C. for about 10-12 h. The apparatus was then cooled to about 50° C., evacuated of hydrogen, and purged several times with nitrogen. Trifluroroacetic acid (3.5 mL) was added to the mixture, then the apparatus was resealed, purged several times with hydrogen, and pressurized to 70 psi with hydrogen. The stirred reaction mixture was heated to 80° C. until the reaction was complete by HPLC (intermediate:product <3%). The reaction mixture was then cooled to room temperature. After filtering the mixture through Celite, the catalyst and molecular sieves were washed with 10 mL aliquots of glacial acetic acid, and the washes were combined with the filtrate. The combined filtrates were concentrated by distillation to yield an oil. The oil was dissolved in ethyl acetate and washed with saturated NaHCO3. The aqueous fraction was extracted with ethyl acetate, then made acidic with concentrated HCl and extracted several times with ethyl acetate. The combined organic fractions were combined and concentrated to a solid. The solid was washed with acetonitrile, filtered, and dried to afford ethyl 6-acetamido-2-chroman-2-carboxylate (about 3.5-4.0 g) as a white solid.

### Example 42

Production of 6-acetamido-2-hydroxymethylchromane

To a solution of ethyl 6-acetamido-2-chroman-2-carboxylate (3.0 g) (Example 41) in dry ether (20 mL) was added dropwise a solution of 1 mL of lithium aluminum hydride (70% in benzene) in about 2 mL of dry ether. The solution was refluxed for about 1 h. An additional 1 mL of lithium aluminum hydride was added to the mixture, and the mixture was refluxed for an additional 1-2 h. The reaction mixture was cooled to 0° C. and the excess hydride was quenched with 1 N H2SO4 (10-12 mL), followed by water (100 mL). The precipitate was filtered and washed with ether. The aqueous phase was separated, extracted several times with 60-mL aliquots of ether. The combined organic extracts were washed with water and dried over anhydrous MgSO4. Evaporation to dryness in vacuo affords 6-acetamido-2-hydroxymethylchromane (about 2.4 g, 90% yield).

### Example 43

Production of 6-acetamido-2-cyanomethylchromane

To a solution of 6-acetamido-2-hydroxymethylchromane (2.0 g) (Example 42) in CH2Cl2 (35 mL) and pyridine (1.5 mL) was added p-toluenesulfonyl chloride (2 g). The mixture was stirred at 25° C. for 36 h, then diluted with ether (20 mL), and washed with water (10 mL). The organic layer was dried over MgSO4 and concentrated in vacuo to give the tosylate(3.4 g).

To a stirred solution of the tosylate in DMSO (20 mL) was added powdered sodium cyanide (80 mg). The mixture was refluxed for 1.5 h under an inert atmosphere, then cooled, diluted with water (50 mL), and extracted with ether (6×100 mL). The combined organic extracts were dried over anhydrous MgSO4 and filtered. The filtrate was concentrated in vacuo and the residue was recrystallized from ether/isopropanol to afford 6-acetamido-2-cyanomethylchromane (1.7 g, 85% yield).

### Example 44

Production of ethyl 6-aminochroman-2-ylacetate hydrochloride

A solution of 6-acetamido-2-cyanomethylchromane (1.5 g) (Example 43) in concentrated hydrochloric acid (25 mL) was stirred vigorously at room temperature for about 6 h. Ethanol (50 mL) was added and the mixture was allowed to stand overnight. The precipitate was filtered, rinsed with ether (50-mL) and dried. Absolute ethanol (25 mL) and HCl (10 mL) were added to the precipitate and stirred for 2 h. Then, concentrated HCl (10 mL) was added to the reaction mixture. The precipitate was recovered and recrystallized twice from ether/isopropanol affording ethyl 6-aminochroman-2-ylacetate hydrochloride (about 1.3 g, 80% yield).

In view of the above description it is believed that one of ordinary skill can practice the invention. The examples given above are non-limiting in that one of ordinary skill in view of the above will readily envision other obvious permutations and variations without departing from the principal concepts. Such permutations and variations are also within the scope of the present invention.

What is claimed is:

1. A process for making a bicyclic compound, or a pharmaceutically acceptable salt of such compound, according to the following formula: 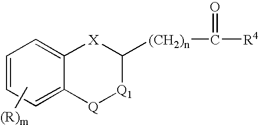
wherein:
n is 0 to 6;
X is O or S;
m is 0 to 4;
R are independently selected from the group consisting of alkyl, alkoxy, lower alkenyl, hydroxy, thio, amino, substituted amino, nitro, halo, and CF₃;
R⁴ is selected from the group consisting of hydroxy, alkoxy, alkenyloxy, halogen, amino, and substituted amino, and
each of Q and Q₁ are independently selected from the —C(—R², —R³)—, wherein each of R² and R³ is independently selected from the group consisting of alkyl, alkoxy, alkenyl, hydroxy, thio, nitro, halo, and CF₃, or R² and/or R³ together with the carbon to which it is attached form a carbonyl group, comprising:
(a) combining a first compound having the formula: 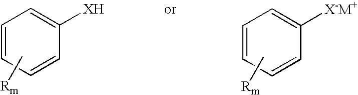
wherein M⁺ is a metal ion; with a second compound having the formula R⁴—Q—Q₁—CH(Z)—(CH₂)ₙ—C(═O)—R¹ wherein Z is halo or OH, optionally in the presence of a Walden catalyst; and
(b) cyclizing the product of (a) to form a bicyclic compound.

2. A process according to claim 1, wherein the Walden catalyst in (a) is selected from the group consisting of PCl₅, PBr₃, PI₃, PF₃, SOCl₂, KOH and Ag₂O; and (b) comprises use of a basic Walden catalyst or a Friedel Crafts catalyst.

3. A process according to claim 2, wherein the catalyst in (b) is selected from the group consisting of AlCl₃, NaOH, KOH, sodium carbonate, and potassium carbonate.

4. A process according to claim 1, wherein the R⁴—Q—Q₁—CH(Z)—(CH₂)ₙ—C(═O)—R¹ used is substantially enantiomerically pure with respect to the CH(Z) carbon.

5. A process according to claim 1, wherein the compound formed is a (2S) or (2R) compound, or an enriched (2S>2R) or (2R>S) mixture wherein one of the (2S) or (2R) is present at greater than 70%.

6. A process according to 5 which produces exclusively an (S) or (R) compound prior to cyclizing.

7. A process according to any of claims 1 through 6, wherein X is oxygen, Q. is CH₂; Q is CH₂ or C═O;.

8. A process according to any of claims 1 through 7, R is a 6-amino group and m is 1.

9. A process according to any of claims 1 through 8, wherein n is 1.

10. A process according to any of claims 1 through 9, wherein R⁴ is an ethoxy group.

11. A process according to claim 1, wherein the second compound is a mono or diester of 3-halomalonic acid, or a mono or diester of 3-hydroxymalonic acid; Z is oxygen; m is 0 or 1; and R is amino or nitro.

12. A process according to claim 1, wherein step (a) produces a compound having the formula: 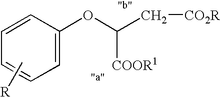
wherein R¹ is hydrogen or alkyl.

13. A process according to claim 12, wherein step (b) comprises
(b1) acylating the compound from step (a) at chain “a” to form an acyl halide;
(b2) displacing the halide with ammonia to form an amido group; and
(b3) performing a Friedel-Crafts cyclization to form a compound having the formula:

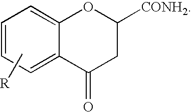

14. A process according to claim 13, further comprising:
hydrolyzing the amido group at the 2-position to form an acid; and
performing a chain extension to form a compound having the formula:

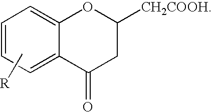

15. A process according to claim 12, wherein step (b) comprises:
(b1) reducing at chain “a” to form a methanol group;
(b2) displacing the methanol hydroxyl group with a cyano group and acidifying to form an acetic acid group;
(b3) performing a Friedel-Crafts cyclization to form a compound having the formula:

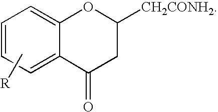

16. A process according to claim 15, further comprising hydrolyzing the amide to form a compound having the formula:

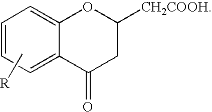

17. A process according to claim 12, wherein step (b) comprises
(b1) acylating the compound from step (a) at both chains “a” and “b” to form acyl halides;
(b2) performing a Friedel-Crafts cyclization to form a compound having the formula:

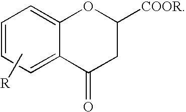

18. A process according to claim 12, wherein step (b) comprises heating the compound from step (a) in the presence of a strong base to form a compound having the formula:

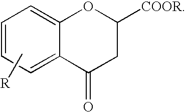

19. A process according to claim 17 or 18, further comprising performing a chain extension to form a compound having the formula:

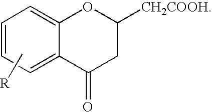

20. A process according to claim 14 or 19; wherein performing a chain extension comprises reducing the acid to an alcohol; displacing the hydroxyl group with a cyano group; and acidifying the cyano group to give the acid.

21. A process according to any one of claims 14, 16, or 19, further comprising exposing the compound to reducing conditions to remove the 4-oxo group.

22. A process according to claim 21, wherein R is nitro and the reducing conditions reduce the nitro group to amino.

23. A process according to any one of claims 13 through 20 wherein m is 0; further comprising nitrating with sulfuric acid and KNO₃ to form a nitro group at the 6-position on the ring system.

24. A process according to claim 23, further comprising exposing the compound to reducing conditions to remove the 4-oxo group and reduce the nitro group to amino, wherein the reduction takes place in one or more steps.

25. A process according to any one of claims 14, 16, 19, 20, 21, 22, 23, or 24, further comprising forming the ester of the acid.

26. A process according to claim 1, wherein
the second compound is a mono or diester of 3-halomalonic acid, or a mono or diester of 3-hydroxymalonic acid;
the first compound is 4-hydroxy-3-methylaniline;
the cyclization in (b) is performed using a strong base.

27. A process according to claim 26, wherein the strong base is potassium t-butylate or n-butyl lithium.

28. A process according to claim 26 or 27, further comprising reducing the compound formed from (b) to remove the 3-oxo group.

29. A process according to any of the foregoing claims wherein the compound formed is enriched in one enantiomer.

30. A process of making a compound according to the formula: 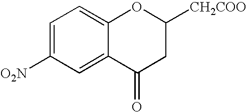
comprising:
reacting a 4-nitrophenoxide compound with a compound having the formula: 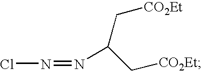
and performing a Friedel-Crafts cyclization reaction.

31. A process of making a compound according to the formula: 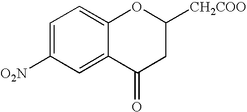
comprising:
reacting a 4-nitrophenoxide compound with a compound having the formula: 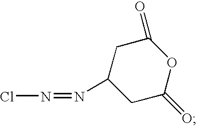
heating the adduct in water to hydrolyze the anhydride; and
performing a Friedel-Crafts cyclization reaction.

32. A process of making a compound according to the formula: 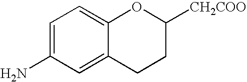
comprising:
exposing a compound produced according to claim 30 or 31 to reducing conditions to remove the 4-oxo group and reduce the nitro group to amino, wherein the reduction takes place in one or more steps.

33. A process for making a compound according to the formula: 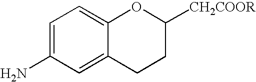
comprising:
(a) reacting 4-nitrophenol and maleic acid in methane sulfonic acid;
(b1) performing a chain extension to lengthen the chain at the 2-position; and
(b2) reducing the 4-oxo and 6-nitro groups;
wherein R¹ is hydrogen or alkyl, and (b1) and (b2) may be performed in either order.

34. A compound having the formula: 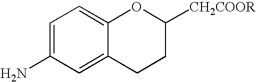
produced by a method according to any one of claims 1-12, 20-22, 24-25, 28-29, or 32-33.

35. A compound having the formula: 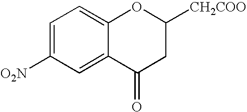
produced by a method according to any one of claims 1-12, 14, 16, 19, 20, 23, 25, 30 or 31.

36. A compound having the formula: 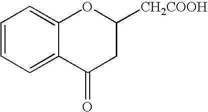
produced by a method according to any one of claims 1-12, 14, 16, 19, 20, or 25.
